(19) United States
(12) Patent Application Publication
(10) Pub. No.: US 2022/0248993 A1
(43) Pub. Date: Aug. 11, 2022

(54) APPARATUS, SYSTEMS AND METHODS FOR ASSESSING INTERNAL ORGANS

(71) Applicant: DIPITY PTY LTD, Kew (AU)

(72) Inventor: Barry Dixon, Kew (AU)

(73) Assignee: DIPITY PTY LTD, Kew (AU)

(21) Appl. No.: 17/619,458

(22) PCT Filed: Jul. 3, 2020
(86) PCT No.: PCT/AU2020/050695

§ 371 Date: Dec. 15, 2021

(30) Foreign Application Priority Data
Jul. 4, 2019 (AU) 2019902373

(51) Int. Cl. A61B 5/1455; A61B 5/00; A61B 5/02; A61B 5/026

(52) U.S. Cl. CPC A61B 5/14552; A61B 5/0059; A61B 5/02014; A61B 5/0261; A61B 2562/162; A61B 5/4064; A61B 5/7246; A61B 2562/0238; A61B 5/14553

(57) ABSTRACT

Apparatus for determining data indicative of blood oxygen levels of internal organs are disclosed. The apparatus comprising: a body comprising a contact surface for engaging with a subject in a vicinity of an internal organ of the subject; the body defining a first recess and second recess, the first and second recesses extending from the contact surface into the body, the second recess being separate from the first recess; a light source comprising a light emitting region located within the first recess and configured to emit light of at least two discreet wavelengths from the first recess of the body onto the internal organ; and a photo-detector comprising a light sensitive region located within the second recess and configured to detect light received at the second recess, wherein the detected light comprises the emitted light reflected from a region of a subject in proximity to an internal organ; wherein the apparatus is configured such that the light emitting region and the light sensitive region are set back from the contact surface by from about 1 mm to about 20 mm and nearest points of the light emitting region and the light sensitive region are separated from one another by from about 4 mm to about 20 mm, such that the detected light is indicative of blood oxygen levels in blood vessels of an outermost surface of the internal organ. Also disclosed are systems comprising the apparatus and methods utilising it for assessing internal organ health of a subject and in particular, but not exclusively, for determining blood oxygen saturation in internal organs of a subject.

### CROSS-REFERENCE TO RELATED APPLICATIONS

This application claims priority from Australian Provisional Patent Application No. 2019902373 filed on 4 Jul. 2019, the disclosure of which is incorporated herein in its entirety by way of reference.

### TECHNICAL FIELD

The present disclosure generally relates to apparatus, systems and methods for assessing internal organ health of a subject. In particular, but not exclusively, the present disclosure relates to apparatus, systems and methods for determining blood oxygen saturation in internal organs of a subject.

### BACKGROUND

Pulse oximetry is used to non-invasively measure absolute arterial circulation oxygen levels (blood oxygen saturation) of the blood of the skin of a subject to thereby provide an indication of the health of the subject. The absolute arterial oxygen levels may be determined by analysing the ratio of intensity of red and near infrared light. The arterial oxygen level can, for example, be obtained by transmitting light through a finger of a subject or by reflecting light from say the forehead skin of the subject and measuring the light captured by a detector. The light is typically produced by a light emitting diode (LED) which is placed directly against the skin of the subject to maximise the light projected into the subject. The signal arises from the blood flow in the skin.

The inventor has now determined that it is possible to accurately determine blood oxygen concentration in the microvasculature of internal organs and assess organ health by utilising an apparatus located on the subject in a vicinity of an internal organ of interest, wherein the light source and photo-detector of the apparatus are set back from a surface of the apparatus in contact with the subject (for example located on the subject's skin) and wherein the light source and photo-detector are spaced-apart to a defined extent, such as from about 5 mm to about 20 mm from the centre of each.

### SUMMARY

Some embodiments relate to an apparatus for determining data indicative of blood oxygen levels of internal organs, the apparatus comprising: a body comprising a contact surface for engaging with a subject in a vicinity of an internal organ of the subject; the body defining a first recess and second recess, the first and second recesses extending from the contact surface into the body, the second recess being separate from the first recess; a light source comprising a light emitting region located within the first recess and configured to emit light of at least two discreet wavelengths from the first recess of the body onto the internal organ; and a photo-detector comprising a light sensitive region located within the second recess and configured to detect light received at the second recess, wherein the detected light comprises the emitted light reflected from a region of a subject in proximity to an internal organ; wherein the apparatus is configured such that the light emitting region and the light sensitive region are set back from the contact surface by from about 1 mm to about 20 mm and nearest points of the light emitting region and the light sensitive region are separated from one another by from about 4 mm to about 20 mm, such that the detected light is indicative of blood oxygen levels in blood vessels of an outermost surface of the internal organ.

The separation between the nearest points of the light emitting region and the light sensitive region may be in the range of about 5 mm to about 15 mm. In some embodiments, the separation is in the range of about 6 mm to about 8 mm.

In some embodiments, the body may further comprise an outer frame defining the contact surface and a cavity; and an inner frame shaped to fit within the cavity, wherein the inner frame defines the first recess and the second recess.

The light source may be configured to emit and sense light comprising light with wavelengths within at least a first wavelength range from about 600 nm to about 750 nm, a second wavelength range from about 855 nm to about 945 nm, and a third wavelength range from about 780 nm to about 820 nm. The photodetector may be configured to emit and sense light comprising at least discrete wavelengths of about 660 nm, about 805 nm, about 895 nm and/or about 940 nm.

Some embodiments of the invention relate to a system for determining blood oxygen levels of internal organs comprising the apparatus described above and a processor, wherein the apparatus and processor are connected to enable transmission of data indicative of blood oxygen levels of internal organs from the apparatus to the processor. The processor may comprise memory, a display and a user interface that are all coupled to the processor. Some embodiments relate to a method of obtaining data indicative of blood oxygen levels of an internal organ of a subject comprising: positioning the apparatus described above on an outer surface of the subject adjacent the internal organ; projecting light from the light source through the outer surface of the subject to the internal organ, wherein the light comprises light at two or more discrete wavelengths; receiving light at a photo-detector of the apparatus, the received light reflected from the internal organ at the two or more discrete wavelengths respectively; and producing a first signal indicative of the intensity of light at the first wavelength and a second signal indicative of the intensity of light at the second wavelength.

In some embodiments, the method comprises locating the apparatus on the scalp of the subject, wherein the internal organ comprises a brain. In some embodiments, the method comprises locating the apparatus adjacent to a region of the skull underlying the scalp, where the region of the skull is relatively thin. The region of the skull may be adjacent to a Sylvian fissure. In some embodiments, the method comprises locating the apparatus in the ear canal of the subject, wherein the internal organ comprises a brain. In some embodiments, the method comprises locating the apparatus on the sternal notch, on supraclavicular spaces, or between ribs of the subject, wherein the internal organ comprises a lung. In some embodiments, the method comprises locating the apparatus below ribs in right upper quadrant or the epigastrium of the subject, wherein the internal organ comprises a liver. In some embodiments, the method comprises locating the apparatus on an abdomen or either of the lower quadrants of the subject, wherein the internal organ comprises intestines. In some embodiments, the method comprises locating the apparatus on a back of the subject, wherein the internal organ comprises a kidney. In some embodiments, the method comprises locating the apparatus over the sternum of the chest or along the left border of the sternum where it meets the ribs, wherein the internal organ comprises a heart. In some embodiments, the method comprises locating the apparatus over a skeletal muscle of the subject, wherein the internal organ comprises the skeletal muscle.

In some embodiments, the method may further comprise: responsive to receiving an instruction indicative of the apparatus being inaccurately positioned relative to the internal organ, repositioning the apparatus relative to the internal organ based on the instruction.

Some embodiments relate to a computer-implemented method of assessing the health of a subject, the method comprising: receiving one or more signals derived from measured light reflected from a region of a subject in proximity to an internal organ at respective distinct wavelengths; determining that at least one waveform of the one or more signals is representative of a signal predominantly associated with the internal organ; and comparing data derived from the at least one waveform with information characteristic of a health condition to assess the health of the subject.

In some embodiments, determining that the at least one waveform is representative of a signal predominantly associated with the internal organ comprises: determining that the at least one waveform corresponds substantially with a venous waveform.

In some embodiments, determining that the at least one waveform is representative of a signal predominantly associated with the internal organ comprises: determining that the at least one waveform comprises an A-wave component, X-wave component, and an Y-wave component corresponding to an A-wave, X-wave and Y-wave of a venous signal. For example, the internal organ may be one of: brain, lung, liver, intestine and a foetal organ.

In some embodiments, determining that at least one waveform is representative of a signal predominantly associated with the internal organ comprises: determining that the at least one waveform is not indicative of an arterial signal derived from a region of skin of the subject.

In some embodiments, determining that at least one waveform is representative of a signal predominantly associated with the internal organ comprises: receiving a further signal derived from the subject substantially simultaneously as the one or more signals, wherein the further signal is derived from measured light reflected from skin of the subject at further wavelength; and determining that a signal peak of the at least one waveform is offset in time from a respective signal peak of a further waveform of the further signal.

The at least one waveform may comprises at least one window corresponding to systolic and diastolic stages of the cardiac cycle. In some embodiments, the method further comprises determining timing of the systolic and diastolic stages of the cardiac cycle associated with the at least one waveform based on the further signal.

In some embodiments, the method further comprises determining an estimate of the diastolic blood oxygen level of the internal organ based on a time offset between the signal peak and the respective signal peak of the further waveform, and a known systolic blood oxygen level. For example, the further waveform of the further signal may be indicative of an arterial pulse of the subject. The further waveform of the further signal may be indicative of a venous signal obtained from a jugular vein of the subject. The further signal may be derived from measured light at wavelength in the range of about 780 nm to about 820 nm, which is sensitive to blood but insensitive to changes in the blood oxygen levels.

In some embodiments, determining that at least one waveform is representative of a signal predominantly associated with the internal organ comprises: determining that the at least one waveform conforms substantially with a template waveform characteristic of the internal organ.

In some embodiments, the method further comprises determining that the at least one waveform is not representative of a signal predominantly associated with the internal organ in response to determining that the at least one waveform is indicative of an arterial signal derived from a region of skin of the subject.

In some embodiments, the one or more signals may comprise a first signal derived from light at a first wavelength and a second signal derived from light at a second wavelength, and the at least one waveform may comprise a first waveform of the first signal and a second waveform of the second signal, and determining that at least one waveform is representative of a signal predominantly associated with the internal organ may comprise: determining a plurality of modified ratio of ratio values across a window of the at least one waveform corresponding to a cardiac cycle, wherein the modified ratio of ratio values are indicative of the blood oxygen level of the internal organ; and determining that the determined plurality of modified ratio of ratio values correspond substantially with characteristic modified ratio of ratio values of the internal organ.

In some embodiments, the internal organ comprises a brain, and determining that one or more signals are predominantly associated with the brain comprises determining that at least one waveform of the one or more signals comprise a first component with a generally increasing signal level at a first rate followed by a second component with a generally decreasing signal level at a second rate that has a smaller magnitude than the first rate.

In some embodiments, determining that one or more signals are predominantly associated with the brain further comprises determining that a pulse onset of the at least one waveform is delayed relative a corresponding pulse onset of an arterial signal obtained from the skin.

In some embodiments, wherein the internal organ comprises a lung, a first signal of the one or more signals is derived from light at a wavelength of around 660 nm, and determining that the first signal is predominantly associated with the lung comprises determining that a first waveform of the first signal corresponds to an inverted pulmonary arterial pressure waveform.

In some embodiments, wherein the internal organ comprises a lung, determining that the one or more signals are predominantly associated with the lung comprises determining that at least one waveform of the one or more signals comprises a component corresponding to a systolic pulse, diastolic pulse and a dicrotic notch.

In some embodiments, wherein the internal organ comprises a liver, and determining that the one or more signals are predominantly associated with the liver comprises determining that at least one waveform of the one or more signals comprises at least one of an X-wave component and a P-wave component.

In some embodiments, wherein determining that one or more signals are predominantly associated with the liver further comprises determining that a pulse onset of the at least one waveform is delayed relative a corresponding pulse onset of an arterial signal obtained from the skin.

In some embodiments, wherein the internal organ comprises intestines, and determining that the one or more signals are predominantly associated with the intestines comprises determining that the at least one waveform of the one or more signals comprises one or more venous wave components, the pulse onset of the one or more venous wave components being delayed relative to corresponding wave components of an arterial signal obtained from the skin of the subject.

In some embodiments, wherein the internal organ comprises a kidney, a first signal of the one or more signals is derived from light at a wavelength of around 895 nm, and determining that the first signal is predominantly associated with the kidney comprises determining that a first waveform of the first signal corresponds to an arterial waveform.

In some embodiments, wherein the internal organ comprises a skeletal muscle, and determining that the first signal is predominantly associated with the skeletal muscle comprises determining that at least one waveform of the one or more signals corresponds to an arterial waveform with a relatively low pulse amplitude.

The method may further comprise assessing the health of the subject based on the comparison and outputting an assessment of the health of the subject. For example, comparing data derived from the at least one waveform with information characteristic of the health condition may comprise comparing one or more components of the at least one waveform to one or more corresponding components of one or more template waveforms, each of the one or more template waveforms being characteristic of the health condition. In some embodiments, comparing data derived from the at least one waveform with information characteristic of the health condition may comprise comparing a first shape of the at least one waveform to a second shape of one or more template waveforms.

In some embodiments, responsive to determining that a V-wave component of the at least one waveform has a larger amplitude than a corresponding V-wave component of a corresponding template waveform of the internal organ, determining that the subject is likely to have heart failure.

In some embodiments, responsive to determining that a first component of the at least one waveform is indicative of an increasing signal level at a first rate, that a second component, following the first component, is indicative of a decreasing signal level at a second rate, and that the magnitude of the first rate is smaller than the second rate, determining that the subject is likely to have relatively high intracranial pressure and/or a brain haematoma.

In some embodiments, wherein the internal organ comprises a brain, and responsive to determining that the at least one waveform does not depict a V-wave or Y-wave component, determining that the subject is likely to have relatively high intracranial pressure.

In some embodiments, wherein the internal organ comprises a brain, and responsive to determining that an AC signal level value of the at least one waveform exceeds a threshold value, determining that the subject is likely to have increased intracranial pressure.

In some embodiments, wherein the internal organ comprises a brain, and responsive to determining that a DC signal level value of the at least one waveform of the one or more signals is less than a threshold value, determining that the subject is likely to have increased intracranial pressure.

In some embodiments, responsive to determining that the at least one waveform comprises an oscillation of about 7 Hz, determining that the subject is likely to have relatively very high intracranial pressure and/or brain haemotoma.

In some embodiments, wherein the one or more signals comprises a first signal associated with a respective first waveform and a second signal associated with a respective second waveform, and wherein the first wavelength is longer than the second wavelength, and the first and second waveforms are representative of signals predominantly associated with a lung, responsive to determining that the first waveform comprises venous waveform characteristics and the second waveform comprises venous waveform characteristics, determining that the health condition comprises a poorly ventilated lung.

In some embodiments, wherein the one or more signals comprises a first signal associated with a respective first waveform and a second signal associated with a respective second waveform, wherein the first wavelength is longer than the second wavelength and the internal organ comprises a lung, responsive to determining that the first waveform comprises a prominent V-wave component and the second waveform does not comprise a prominent V-wave component, determining that the health condition comprises a poorly ventilated lung.

In some embodiments, wherein the internal organ comprises a liver, and responsive to determining that at least one waveform of the one or more signals comprises a prominent P-wave component, determining that the health condition comprises high portal vein blood flow.

In some embodiments, wherein the internal organ comprises a liver, and responsive to determining that at least one waveform of the one or more signals differs from a template waveform representative of a healthy liver by a threshold amount, determining that the health condition comprises any one or more of hepatitis, cirrhosis and right heart failure.

In some embodiments, wherein the internal organ comprises a heart, and the method further comprises, responsive to determining that the at least one waveform conforms substantially to template waveform characteristic of abnormal movements of the heart, determining that the heart is damaged due to a myocardial infarct or heart failure.

In some embodiments, wherein the internal organ comprises a heart, and the method further comprises analysing the one or more signals to determine timing in the cardiac cycle of the heart chambers' contractions and relaxations.

In some embodiments, wherein the one or more signals comprise a first signal derived from light at a first wavelength and a second signal derived from light at a second wavelength, and the at least one waveform comprises a first waveform of the first signal and a second waveform of the second signal, the method further comprises: determining a plurality of modified ratio of ratio values across a window of the at least one waveform, wherein the window corresponds with systolic and diastolic stages of the cardiac cycle, and wherein the modified ratio of ratio values are indicative of the blood oxygen level of the internal organ. Determining a plurality of modified ratio of ratio values across the window the at least one waveform corresponding to a cardiac cycle may comprise sampling oxygen levels at a relatively high rate across the window.

In some embodiments, comparing data derived from the at least one waveform with information characteristic of the health condition comprises responsive to determining that the determined plurality of modified ratio of ratio values deviates from characteristic ratio of ratio values of the internal organ, determining that the internal organ is potentially unhealthy.

In some embodiments, the method further comprises: receiving third and fourth signals derived from the subject substantially simultaneously as the one or more signals, wherein the third and fourth signals are derived from measured light reflected from skin of the subject at a respective third and fourth distinct wavelengths; and determining a plurality of modified ratio of ratio values across a window of third and fourth waveform associated with the respective third and fourth signals, wherein the window corresponds with systolic and diastolic stages of the cardiac cycle, and wherein the modified ratio of ratio values are indicative of the blood oxygen level of the skin. In some embodiments, the method comprises comparing the plurality of modified ratio of ratio values derived from the skin of the subject with the plurality of modified ratio of ratio values derived from the internal organ of the subject and responsive to determining that the modified ratio of ratio values differ across the window corresponding to the cardiac cycle, determining that the first and second signals are representative of signals predominantly associated with the internal organ.

For example, the modified ratio of ratios may be calculated by:

$$R ( t ) = \frac{( \frac{A {C}_{1} ( t )}{{I}_{1} ( {t}_{0} )} )}{( \frac{A {C}_{2} ( t )}{{I}_{2} ( {t}_{0} )} )}$$

where the first wavelength is shorter than the second wavelength and AC₁ (t) is the change in the signal level of the first signal from I₁ at time t, AC₂(t) is the change in the signal value of the second signal from I₂ at time t, and I₁(t₀) is the signal value of the first signal at time t₀ used as a first normalisation factor, and I₂(t₀) is the signal value of the second signal at time t₀ used as the second normalisation factor for the longer wavelength, also taken at time t₀. The time t₀ may be the time of the peak light intensity signal value (I₁) of the first signal and the normalisation factor (I₂) of the second signal may be the light intensity signal value of the second signal also at time t₀. The time t₀ at which the normalisation factors are determined may be at a time after time t. In some embodiments, the normalisation factor (I₁) and normalisation factor (I₂) of the modified ratio of ratios R may be calculated using the respiratory oscillations to account for signal value changes as a result of respiratory cycles of the subject, wherein t₀ is defined by the time point of the peak signal value at the start of each respiratory oscillation for each wavelength. In some embodiments, the method further comprises averaging the determined modified ratio of ratio values using the cardiac oscillations across the phase of the respiratory cycle in which they occurred to determine the average modified ratio of ratios level for one or more of inspiration, inspiratory pause, expiration and the expiratory pause phases of the respiratory cycle.

In some embodiments, the method further comprises determining a tissue oxygen level value of the internal organ based on the maximum modified ratio of ratio values over the systolic and diastolic phases of a cardiac cycle. For example, the method may comprise determining the end of diastole and the temporal point of the maximum R values based on determination of an A-wave component of the first and second waveforms. The method may comprise determining the tissue oxygen level value of the internal organ based on the rate of change of modified ratio of ratio values over the systolic and diastolic phases of a cardiac cycle.

The method may comprise comparing the blood oxygen level with a threshold level; and responsive to determining that the blood oxygen level is less than the threshold level determining that the subject is suffering an adverse health condition.

In some embodiments, the method may comprise comparing the blood oxygen level with a threshold level; and responsive to determining that the blood oxygen level is greater or lower than the threshold level determining that the subject has an increased intracranial pressure, wherein the internal organ comprises a brain.

In some embodiments, the method may comprise analysing a fall in blood oxygen level during a diastolic cardiac phase to determine clinical information.

In some embodiments, the method may comprise determining an oscillation in minimum blood oxygen levels in organs of the body over a plurality of respiratory cycles to assess the oxygen exchange in the lungs.

In some embodiments, wherein the internal organ comprises a lung, the method may comprise determining blood oxygen levels throughout the first and second waveforms; and determining an indication of the systemic arterial oxygen level based on the peak blood oxygen level during a diastolic cardiac phase.

In some embodiments, wherein the internal organ comprises a lung; and the method further comprises: determining a maximum blood oxygen level to provide an indication of the lung function.

In some embodiments, wherein the internal organ comprises a lung; and the method further comprises: determining a minimum blood oxygen level to estimate of mixed venous blood oxygen values for blood entering the lung.

In some embodiments, wherein determining that at least one waveform of the one or more signals is representative of the signal predominantly associated with the internal organ comprises determining that a waveform of the blood oxygen levels conforms significantly with a template blood oxygen waveform characteristic of the internal organ.

In some embodiments, the method may comprise receiving at least one further signal, the further signal being derived from received light reflected from a further region of the subject in proximity to another internal organ at respective distinct wavelength; determining that at least one further waveform of the at least one further signal is representative of a further signal predominantly associated with the further internal organ; and comparing data derived from the at least one further waveform with information characteristic of a health condition to diagnose systemic or regional disorders of the subject.

In some embodiments, the method may comprise: receiving at least one further signal, the further signal being derived from received light reflected from skin the subject at respective distinct wavelength; determining that at least one further waveform of the at least one further signal is representative of a further signal predominantly associated with skin of the subject; comparing data derived from the at least one further waveform with information characteristic of a health condition to diagnose systemic or regional disorders of the subject.

In some embodiments, assessing health comprises monitoring blood flow in the internal organ based on a comparison of the shape and/or amplitude of the at least one waveform with one or more template waveforms. The at least one or more waveform may be associated with a first signal at a wavelength of 805 nm.

In some embodiments, the method may comprise responsive to determining that the at least one waveform is substantially similar to an arterial blood pressure waveform, determining that the arterial blood pressure of the subject is much greater than the central venous pressure and that blood flow is high.

In some embodiments, wherein when the organ being targeted is the liver, the method may comprise responsive to determining that the at least one waveform comprises a P wave component having a relatively high amplitude, determining that there is very high portal vein blood flow and or a hypoxic liver.

In some embodiments, the method may comprise responsive to determining that the at least one waveform comprises an exaggerated venous waveform, determining that venous pressure is high and organ blood flow low. For example, the exaggerated venous waveform may comprise a high amplitude V wave component and optionally a high amplitude A wave component and for example, it may be determined that the subject has one or more of: raised central venous pressure levels, fluid overload and heart failure.

In some embodiments, the method comprises receiving information indicative of the internal organ being targeted. For example, determining that at least one waveform of the one or more signals is representative of the signal predominantly associated with the internal organ may be at least partially based on the received information indicative of the internal organ being targeted. The information characteristic of the health condition may be at least partially based on the received information indicative of the internal organ being targeted.

Some embodiments relate to a computer-implemented method of assessing breathing of a subject, the method comprising: receiving one or more signals derived from measured light reflected from a region of a subject in proximity to an internal organ at respective distinct wavelengths; calculating a statistical measure of the intensity of the one or more signals; and determining that the statistical measure varies over time and associating the variation with a breathing pattern of the subject. The method may further comprise outputting a control instruction to control a mechanical ventilator based on the determined breathing pattern. The internal organ may be or comprises any one of: brain, liver, lung, intestine, heart, foetus.

Some embodiments relate to a system for assessing health of a subject, the system comprising: one or more processors; and memory including computer executable instructions, memory being coupled to the one or more processors; wherein the one or more processors are configured to execute the computer executable instructions to cause the system to perform any one of the described methods

Some embodiments relate to a computer program comprising instructions which, when executed by one or more processors, cause the one or more processors to perform any one of the described methods.

Some embodiments relate to a system for assessing a health condition of a subject comprising: any one of the described apparatus for determining blood oxygen levels; memory comprising computer executable instructions; a processor coupled to the memory and configured to execute the computer executable instructions to perform any one of the described methods. In some embodiments, the system comprises a second apparatus for determining blood oxygen levels, the second apparatus comprising a second light source and a second photodetector configured to receive a further signal indicative of an arterial pulse in the skin of the subject, and configured to provide the further signal to the processor. In some embodiments, the system comprises one or more second apparatus for determining blood oxygen levels of the internal organ, the one or more second apparatus comprising a second light source and a second photodetector configured to receive a further signal indicative of a signal from the internal organ, and configured to provide the further signal to the processor. In some embodiments, the system comprises one or more second apparatus for determining blood oxygen levels of one or more second internal organs, the one or more second apparatus comprising a second light source and a second photodetector configured to receive a further signal indicative of a signal from the second internal organ, and configured to provide the further signal to the processor.

In some embodiments, the apparatus may be coupled to catheters to be placed in the body of the subject.

Some embodiments relate to a method of obtaining data indicative of intra-cranial pressure of a subject comprising: positioning a light source of any one of the described apparatus in a spaced apart manner relative to a cranium of the subject adjacent to a sulcus of a brain of the subject; projecting light from the light source through the cranium of the subject to the sulcus, wherein the light comprises light at one or more discrete wavelengths; receiving light at a photodetector of the apparatus, the received light reflected from cerebrospinal fluid in the sulcus at the one or more discrete wavelengths; producing one or more signals indicative of the intensity of light at the one or more discrete wavelength; and providing the one or more signals to the described system to allow for determination of raised intra-cranial pressure of the subject based on a waveform of at least one of the one or more signals.

The method may comprise determining raised intra-cranial pressure of the subject based on a waveform of at least one of the one or more signals and responsive to determining that the pulse waveform comprises one or more oscillations, determining raised intra-cranial pressure based on one or more of pattern, amplitude and frequency of the one or more oscillations.

In some embodiments, determining raised intra-cranial pressure may comprise determining that the pulse waveform comprises oscillations similar to the waveform of an intra-cranial pressure trace and the pulse onset being before a corresponding wave components of an arterial signal obtained from the forehead skin.

Some embodiments relate to a method of assessing a health condition of a foetus within a subject, the method comprising: receiving one or more signals, the one or more signals being derived from received light reflected from a region of the foetus in proximity to the internal organ at respective discrete wavelengths; receiving at least one further signal, the at least one further signal being derived from received light reflected from a region of the subject at a further discrete wavelength; determining that at least one waveform of the at least one signal is representative of a signal predominantly associated with the internal organ based on a comparison of waveforms comprising comparing the at least one waveform with at least one further waveform of the at least one further signal; comparing data derived from at least one of the at least one waveform with information characteristic of a health condition to assess the health of the foetus.

The method may comprise locating any one of the described apparatus (a first apparatus) on an outer surface of the subject adjacent to an internal organ of the foetus to detect the first signal; and locating any one of the described apparatus (a second apparatus) on any one of: a forehead, a finger, an ear, and a nose, of the subject to detect the second signal. The comparison of waveforms may comprise determining an offset in time between peak signal levels. The method may comprise locating the first apparatus on the abdomen of the subject. The method may comprise intra-vaginally locating the first apparatus in the subject.

### BRIEF DESCRIPTION OF DRAWINGS

Embodiments are described in further detail below, by way of example, with reference to the accompanying drawings briefly described below. Like reference labels in the drawings indicate like features.

FIG. 1 is a isometric view of an apparatus for obtaining data relating to blood oxygen levels of an internal organ of a subject according to some embodiments;

FIG. 2(a) is a side view of the apparatus of FIG. 1;

FIG. 2(b) is a top view of the apparatus of FIG. 1;

FIG. 3 is a cross-sectional view of the apparatus of FIG. 1 along line A-A;

FIG. 4 is an exploded isometric view of the apparatus of FIG. 1;

FIG. 5 is a schematic diagram of a system for obtaining data relating to blood oxygen levels from an internal organ of a subject according to some embodiments;

FIG. 6 is a flow diagram for a method of obtaining data indicative of the blood oxygen level of an internal organ of a subject according to some embodiments;

FIG. 7(a) is a plot of first and second signals derived from detected light reflected from the brain of a healthy human subject;

FIG. 7(b) is a plot of the calculated modified ratio of ratios from the first and second signals of FIG. 7(a);

FIG. 8(a) is a plot of third and fourth signals derived from detected light reflected from the forehead skin of a healthy human subject;

FIG. 8(b) is a plot of fifth and sixth signals derived from detected light reflected from an internal jugular vein of the healthy subject, which was obtained substantially simultaneously with the detected light for the plot of FIG. 8(a);

FIG. 8(c) is a plot of first and second signals derived from detected light reflected from the brain of the healthy subject, which was obtained simultaneously with the detected light for the plot of FIG. 8(a);

FIG. 9 is a flow diagram for a method of assessing the health of a subject according to some embodiments;

FIG. 10(a) is a plot of first and second signals derived from detected light reflected from a well ventilated lung of a healthy human subject;

FIG. 10(b) is a plot of the calculated modified ratio of ratios from the first and second signals of FIG. 10(a);

FIG. 11 is a plot of first and second signals derived from detected light reflected from the liver of a human subject;

FIG. 12(a) is a plot of third and fourth signals derived from detected light reflected from the skin of the nose of an ovine subject with a relatively high intra-cranial pressure;

FIG. 12(b) is a plot of first and second signals derived from detected light reflected from the brain of the ovine subject, which was obtained simultaneously with the reflected light from the nose skin for the plot of FIG. 12(a);

FIG. 13(a) is a plot of third and fourth signals derived from detected light reflected from the skin of the nose of an ovine subject with a relatively very high intra-cranial pressure;

FIG. 13(b) is a plot of first and second signals derived from detected light reflected from the brain of the ovine subject, which was obtained simultaneously with the detected light for the plot of FIG. 13(a);

FIG. 14(a) is a plot of third and fourth signals derived from detected light reflected from the skin of the nose of an ovine subject with a relatively extreme high intra-cranial pressure;

FIG. 14(b) is a plot of first and second signals derived from detected light reflected from the brain of the ovine subject, which was obtained simultaneously with the detected light for the plot of FIG. 14(a);

FIG. 15(a) is a plot of third and fourth signals derived from detected light reflected from the forehead skin of a supine healthy human subject;

FIG. 15(b) is a plot of first and second signals derived from detected light reflected from a poorly ventilated lung of the otherwise healthy human subject, which was obtained simultaneously with the detected light of the plot of FIG. 15(a);

FIG. 15(c) is a plot of the calculated modified ratio of ratios from the first and second signals of FIG. 15(b);

FIG. 16 is a plot of first and second signals derived from detected light reflected from the intestines of a healthy human subject;

FIG. 17 is a plot of a calculated modified ratio of ratios of first and second signals derived from detected light reflected from the brain of 3 human subjects under different levels of systemic hypoxia against the corresponding blood oxygen level determined by collecting blood from the internal jugular vein;

FIG. 18 is a plot of a calculated modified ratio of ratios of first and second signals derived from detected light reflected from the brain of an ovine subject under different levels of brain hypoxia due to reduced blood flow to the brain, against the corresponding blood oxygen level determined by collecting blood from the saggital sinus vein;

FIG. 19 is a plot of the calculated modified ratio of ratios averaged over each pulse from waveforms of respective first and second signals derived from detected light reflected from the brain of an ovine subject following an injection of blood into the brain to raise intracranial pressure and disturb brain blood flow;

FIG. 20(a) is a plot of third and fourth signals derived from detected light from an internal jugular vein of a healthy human subject over a time period long enough to include several respiratory cycles;

FIG. 20(b) is a plot of first and second signals derived from detected light reflected from the brain of the subject, which was obtained simultaneously with the detected light for the plot of FIG. 20(a);

FIG. 20(c) is a plot of the calculated modified ratio of ratios from the first and second signals of FIG. 20(b);

FIG. 21 is a plot of first and second signals derived from detected light reflected from the lung of a human subject while breathing and then holding their breath;

FIG. 22 is a plot of two signals from light at a first and second wavelength reflected from the brain of a human subject with the sensor placed in the ear canal of the subject; and

FIG. 23 (a) shows a plot of the percentage of successful brain pulse detections using an apparatus with lateral separation of the light source and photo-detector centres of 10 mm, 15 mm, 20 mm and 40 mm, in the case where the distance between the light source and photo-detector and the contact surface of the apparatus is zero (i.e. light source and photo-detector are located in the skin of the subject, as is the arrangement with conventional light oximeter devices).

FIG. 23 (b) shows a bar graph of the percentage of successful brain pulse detections using an apparatus with lateral separation of the light source and photo-detector centres of 10 mm, 15 mm and 20 mm, in the case where the distance between the light source and photo-detector and the contact surface of the apparatus is constant at 10 mm (that is, there is a 10 mm separation of the light source and photo-detector from the skin of the subject).

FIG. 23 (c) shows a bar graph of the percentage of successful brain pulse detections using an apparatus with lateral separation of the light source and photo-detector centres constant at 15 mm, and the distance between the light source and photo-detector and the contact surface of the apparatus is varied between 0 mm, 5 mm, 10 mm, 15 mm and 20 mm (that is, the separation of the light source and photo-detector from the skin of the subject is varied between 0, 5, 10, 15 and 20 mm).

FIG. 24 shows a bar graph of the percentage of successful brain pulse detections using an apparatus with lateral separation of the light source and photo-detector centres of 10 mm, where the distance between the photo-detector and the contact surface of the apparatus is fixed at 10 mm and the distance between the light source and the contact surface of the apparatus varied from 15 mm and 20 mm (i.e. 5 mm and 10 mm offset from the light source, respectively).

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

Reference within this specification to prior patent documents or technical publications is intended to constitute an incorporation of the subject matter of such prior publications within the present specification in their entirety, by way of reference.

Described embodiments relate generally to apparatus, systems and methods for assessing internal organ health of a subject.

Monitoring of internal organs, such as the brain following acute brain injury often relies on invasive techniques such as intra-cranial pressure monitoring, intra-parenchymal oxygen sensors and jugular bulb venous catheters. These approaches carry risks, are technically challenging, expensive and can detect deteriorations late (Barone D G, Czosnyka M., ScientificWorldJournal. 2014, 2014:795762, the disclosure of which is incorporated herein in its entirety by way of reference).

Non-invasive monitors of brain oxygen levels, such as cerebral oximeters, exist which analyse the scattering of near infrared light have been proposed to study the brain. However, cerebral oximetry has not found a significant role in clinical applications, with studies demonstrating inconsistent results. See for example, Schneider A, et al., Acta Paediatr, 2014,103(9):934-938; Steppan J, Hogue C W, Jr., Best Pract Res Clin Anaesthesiol, 2014, 28(4):429-439; and Lund A, Secher N H, Hirasawa A, et al., Scand J Clin Lab Invest, 2016, 76(1):82-87 (the disclosures of each of which are incorporated herein in their entirety by way of reference). Measurements of oxygen saturation may also take up 10 to 15 seconds to make which limits the amount of information provided. In addition, these monitors provide no information on the pulse shape which represents blood flow in the organ.

In earlier published International Patent Publication No. WO2008/134813 (the disclosure of which is incorporated herein in its entirety by way of reference) the present inventor described a non-invasive method to directly measure blood oxygen saturation (such as central venous and mixed venous blood oxygen saturation) by placing a light oximeter device on the skin over deep vascular structures. As noted above, pulse oximetry, using red and infrared light sources, is an established technique to measure haemoglobin oxygen saturation of blood vessels in the skin. Deoxyhaemoglobin (Hb) absorbs more of the red band while oxyhaemoglobin absorbs more of the infra-red band. In the earlier International Patent Publication preferred wavelengths of red light of from about 620 nm to about 750 nm and of infra-red light of from about 750 nm to about 1000 nm were disclosed. In pulse oximetry light is first transmitted through the tissues and the intensity of the transmitted or reflected light is then measured by the photo-detector. The pulse oximeter determines the AC (pulsatile) component of the absorbance at each wavelength and the amount of the red and infrared AC components is determined, which is indicative of the concentration of oxyhaemoglobin and deoxyhaemoglobin molecules in the blood. The ratio of oxygenated haemoglobin to total haemoglobin indicates the oxygen saturation of the blood.

In WO2008/134813 the present inventor demonstrated that by utilising the pulsatile nature of the deep vascular structures to generate a plethysmographic trace it is possible to accurately locate the emitter and receiver elements to optimise the signal detected and to thereby do away with the need for concurrent ultrasonography and measurements from more than one location. The individuality of the plethysmography in the technique described was used to identify that the signal is arising from the vascular structure of interest and to filter out signals arising from other interfering chromophores, such as small blood vessels and surrounding tissues.

In WO2008/134813 the present inventor demonstrated that by utilising the pulsatile nature of the deep vascular structures to generate a plethysmographic trace it is possible to accurately locate the emitter and receiver elements to optimise the signal detected and to thereby do away with the need for concurrent ultrasonography and measurements from more than one location. The individuality of the plethysmography in the technique described was used to identify that the signal is arising from the vascular structure of interest and to filter out signals arising from other interfering chromophores, such as small blood vessels and surrounding tissues.

In International Patent Publication No. WO2012/003550 (the disclosure of which is incorporated herein in its entirety by way of reference) the present inventor determined that improvements in accuracy and reliability of blood oxygen saturation determination by oximetry from deep vascular structures can be made by adopting one or more of (a) selecting optimal wavelengths for determination of light absorption by haemoglobin in the blood (for example from about 1045 nm to about 1055 nm and from about 1085 nm to about 1095 nm); (b) locating the oximetry emitter and receiver elements within the external auditory canal of the patient; (c) increasing distance between emitter and receiver elements up to a threshold level of about 60 mm; and (d) angling the emitter element at an angle of approximately 450 relative to the angle of the receiver element.

It is of clinical value to monitor microvascular blood oxygen levels and blood flow in internal organs of patients as disorders may lead to organ failure and lead to death of patients. Early warning of impending organ failure may enable earlier intervention and thereby reduce patient morbidity and mortality rates.

The tissue oxygen levels of the internal organ may be indicative of the internal organ health. Monitoring the tissue oxygen levels for low tissue oxygen levels may therefore provide early indication of pending organ failure. Analysis of reflected light from a region of an internal organ, such as the brain, liver, lung, kidney, intestine and heart, of a subject may provide an indication of microvascular blood oxygen levels in blood vessels associated with the internal organ which may provide valuable clinical information about oxygen transfer to the internal organ, the tissue oxygen levels of the internal organ and accordingly, the health of a subject.

Some embodiments relate to apparatus, systems and methods for assessing the health of a subject based on at least one signal derived from received light reflected from a region of a subject in proximity to an internal organ. The inventor has recognised that in order to ensure accurate assessment of the health of a subject, it is important to ensure that the at least one signal being considered is in fact indicative of light reflected from the internal organ and not the overlying skin. Accordingly, described embodiments involve determining that a waveform of the at least one signal is representative of a signal that is in fact associated with the internal organ. The described embodiments may involve determining that the waveform of the at least one signal is dominated by or predominantly associated with received light reflected from the internal organ.

Some embodiments relate to apparatus, systems and methods for determining more suitable or optimal placement of a sensor with respect to an internal organ to obtain a signal predominantly associated with received light reflected from the internal organ. For example, responsive to determining that a received signal is not predominantly associated with received light reflected from the internal organ, a processor of a system may be configured to output an instruction for relocation or positioning of an apparatus comprising the sensor relative to the internal organ being targeted. The instruction may be effective to cause automatic repositioning of the apparatus or may instruct an operator to reposition the apparatus. In some embodiments, the instruction may comprise indicative distances or coordinates which may be used to relocate the apparatus relative to the internal organ being targeted.

Some embodiments relate to apparatus, systems and methods for determining by a processor of the system, that a received signal may not be arising from or predominantly associated with received light reflected from an internal organ of interest, but is instead a contaminated signal arising from or predominantly associated with the skin.

For example, the waveform may be compared to one or more template waveforms, characteristic of a typical or healthy internal organ, to determine if the waveform is sufficiently similar to the template waveforms to be determined as being associated with a signal dominated by light reflected from the internal organ. For some internal organs, a pulse with venous characteristics in the signal is expected and accordingly, the waveform may be compared with a typical or measured venous waveform to determine that the signal is predominantly associated with the internal organ. In some cases, it is expected that a skin signal or other arterial signal taken from the subject at the same time as the signal would produce a waveform that is earlier than the waveform of the signal predominantly associated with the internal organ, which may also be employed to determine that the signal is predominantly associated with the internal organ. In some embodiments a modified ratio of ratios calculation is performed across a window of the waveform corresponding to a cardiac cycle or pulse and the results are compared with characteristic values of a typical or healthy internal organ to make the determination. In this case, at least two signals derived from received light reflected from a region of a subject at two different wavelengths would be required to perform the modified ratio of ratios calculation.

Once it has been determined that the at least one signal is representative of a signal predominately associated with the internal organ, data derived from the waveform may be employed to assess the health of the internal organ, and accordingly, of the subject. For example, data derived from the waveform may be compared with information characteristic of a health condition to assess the health of the subject. For example, such health conditions may include increased intra-cranial pressure, respiratory disorders, liver failure, heart failure (such as a leaky heart valve), brain haematoma, intestinal ischaemia and/or poorly ventilated lungs, and/or health conditions associated with movement of the internal organs.

In some embodiments, a modified ratio of ratios calculation is performed on the data derived from at least two waveforms derived from the internal organ to make the health assessment.

Some embodiments relate to apparatus, systems and methods for assessing the health of a subject based on a signal derived from received light reflected from a region of a subject in proximity to an internal organ, wherein the signal's pulse shape and amplitude is predominately indicative of the temporal blood flow changes over the duration of the pulse of blood in the organ's microcirculation. This may be achieved, for example, by subjecting the internal organ to light at a wavelength of about 780 nm and 820 nm, and preferably, 805 nm, as light at this wavelength is absorbed to the same extent regardless of oxygen saturation levels. Data derived from a waveform corresponding to the signal predominately indicative of blood flow in the internal organ may be used to make health assessments based on the nature of blood flow in the organ and the circulatory system.

Some embodiments relate to apparatus, systems and methods for obtaining data relating to any one or more of: microvascular blood oxygen levels, microvascular blood pulse shape and amplitude, and also movement of internal organs of a subject. Some embodiments described advantageously enable such data to be non-invasively obtained or extracted from light signals reflected by internal organs with minimal contributions from light reflected by the skin of the subject. The described embodiments may therefore enable health assessment of the internal organ, and accordingly the subject, to be performed, including determining the absolute microvascular blood oxygen saturation, microvascular blood pulse shape and amplitude and also movement of internal organs of the subject.

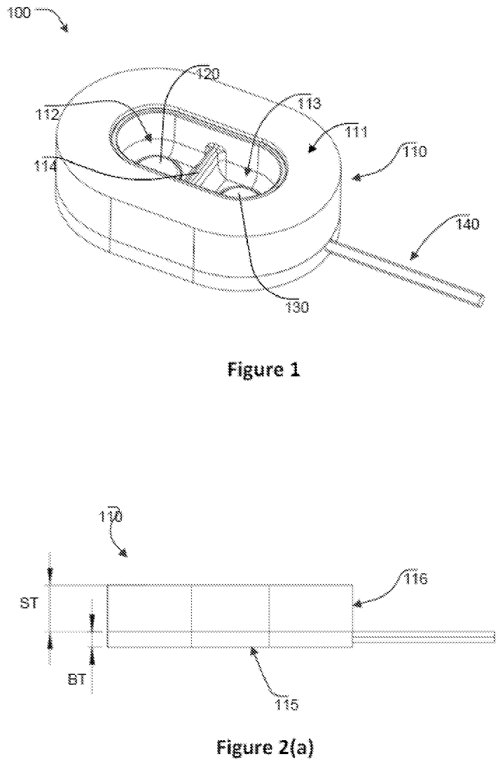

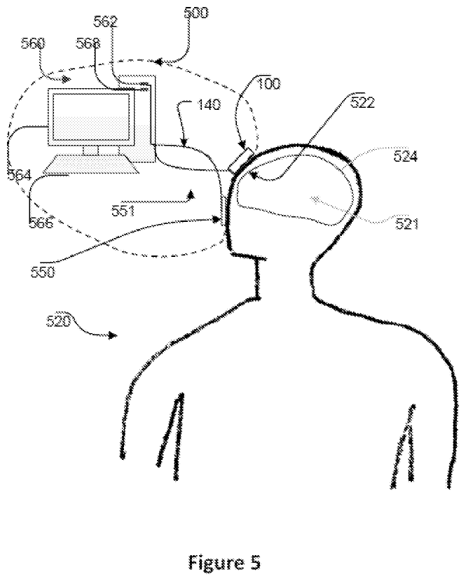

Referring to FIGS. 1, 2(a), 2(b), 3 and 4, an apparatus 100 for determining data indicative of blood oxygen levels of internal organs is shown. The apparatus 100 comprises a body 110 that comprises a contact surface 111 for engaging with a subject 520 (see FIG. 5) in a vicinity of an internal organ of the subject 520 (e.g. a brain 521) that is to be targeted.

The body 110 of the apparatus 100 defines a first recess 112 and second recess 113. The first and second recesses 112, 113 extend from the contact surface 111 into the body 110 and the second recess 113 is separate from the first recess 112.

The apparatus 100 is configured to receive alight source 120 in the first recess 112. The light source 120 is configured to emit light from the first recess 112 of the body 110 onto the internal organ. The apparatus 100 is configured to receive a photo-detector 130 in the second recess 113. The photo-detector is configured to receive or detect light received at the second recess 113. The received light comprises light emitted by the light source 120 that has interacted with the internal organ. For example, the received light may comprise at least a portion of the light emitted by the light source 120 that has been reflected and/or scattered by the internal organ. The wavelength and/or intensity of the received light may be somewhat modified relative to that of the emitted light, for example as a result of light absorption and/or scattering by body tissues/organs. Throughout this specification and the accompanying claims it is to be understood that reference to light reflection or reflected light does not infer that the emitted and reflected light are of the same wavelength and/or intensity. The apparatus 100 is configured such that the light source 120 and the photo-detector 130 are set back from the contact surface 111 (such as for example by from about 1 mm to about 20 mm, such as from about 5 mm to about 15 mm or about 6 mm to about 12 mm, about 7 mm to about 10 mm, or about 7 mm, 8.5 mm or 10 mm) and are spaced-apart from one another by a relatively small amount (e.g. separation S) such that the received or detected light is indicative of blood oxygen levels in blood vessels of the internal organ (e.g. veins on the surface 524 or the microvasculature of the brain 521).

The apparatus 100 is different from standard cerebral oximetry monitors as the light source 120 and photo-detector 130 are not in contact with the skin and have a small separation distance from each other (contrary to existing teaching in the art). The present inventor has determined that these modifications reduce light scatter through the skin and maximise the light reaching the photo-detector 130 that has travelled through at least part of the organ. Thus overcoming the major limitation of existing cerebral oximeters, which derive much of their signal from the skin rather than the underlying organ. In addition, by our knowledge of the expected pulse waveform and oxygen levels of the organ (which differ markedly from skin) we can also confirm that the signal is arising from the organ of interest. Standard cerebral oximeters cannot confirm the source of their signal.

Existing cerebral oximeters have a separation of at least 40 mm between the light source and photo-detector as it is considered important to have a larger spacing to enable detection of light that is reflected from deep within the brain. The inventor has, however, found that a much shorter separation provides an improved signal arising from the organ with a reduction in the skin signal.

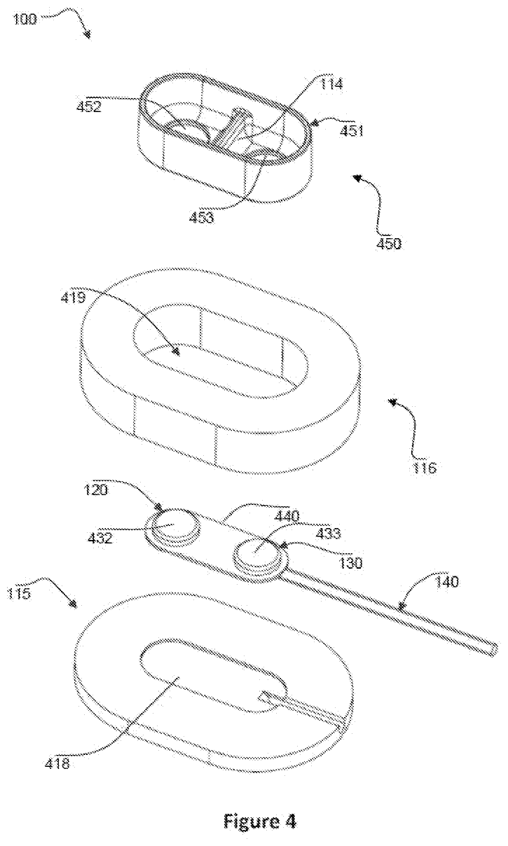

The light source 120 may comprise a light emitting region (shown as 432 in FIG. 4) and the photo-detector 130 may comprise a light sensitive region (shown as 433 in FIG. 4). In the embodiment shown in FIG. 4 the light source 120 and the photo-detector 130 each have a body that sits beneath the light emitting region 433 and light sensitive region, respectively. In some embodiments, such as depicted in FIG. 1, the whole or substantially all of the light source 120 that faces towards the contact surface 111 is light emitting region (not shown) and the whole or substantially all of the photo-detector 130 that faces towards the contact surface 111 is light sensitive region (not shown). For this reason, the light source 120 and light emitting region (not shown) from the perspective depicted in FIG. 1 are indistinguishable from one another, as are the photo-detector 130 and the light sensitive region (not shown). The light emitting region and light sensitive region may be encapsulated by a protective structure. A separation S between the nearest points of the light emitting region and the light sensitive region may be in the range of about 4 mm to about 20 mm. In some embodiments, the separation S is in the range of about 4 mm to about 12 mm, about 5 mm to about 10 mm or about 6 mm to about 8 mm. The separation S may, for example, be about 7 mm.

The centre of the light source 120 may be spaced-apart from the centre of the photo-detector 130 by a distance X of about 20 mm or about 15 mm. In the embodiment shown in FIG. 1 the centre of the light source 120 is also the centre of the light emitting region and the centre of the photo-detector 130 is also the centre of the light sensitive region, but in other embodiments there is body of the light source 120 surrounding the light emitting region and body of the photo-detector 130 surrounding the light sensitive region. For example, in one aspect centre points of the light source 120 (or light emitting region) and the photo-detector 130 (or light sensitive region) are separated by from about 10 mm to about 20 mm, such as about 15 mm, and the separation S between the nearest peripheries of the light source 120 and the photo-detector 130 is from about 4 mm to about 20 mm, such as from about 6 mm to about 8 mm or about 7 mm. In some embodiments the diameter of the light source 120 and the photo-detector 130 is about 8 mm in each case.

The light emitting region of the light source 120 may be set back from the contact surface 111 by a spacing Y. The body 110 of the apparatus may therefore assist in spacing the light emitting region from the subject 520 when the contact surface 111 engages the subject 520.

In some embodiments, the photo-detector 130 may comprise alight sensitive region (not shown) that is set back from the contact surface 111 by the spacing Y. The body 110 of the apparatus 100 may therefore assist in spacing the light sensitive region from the subject 520 when the contact surface 111 engages the subject 520.

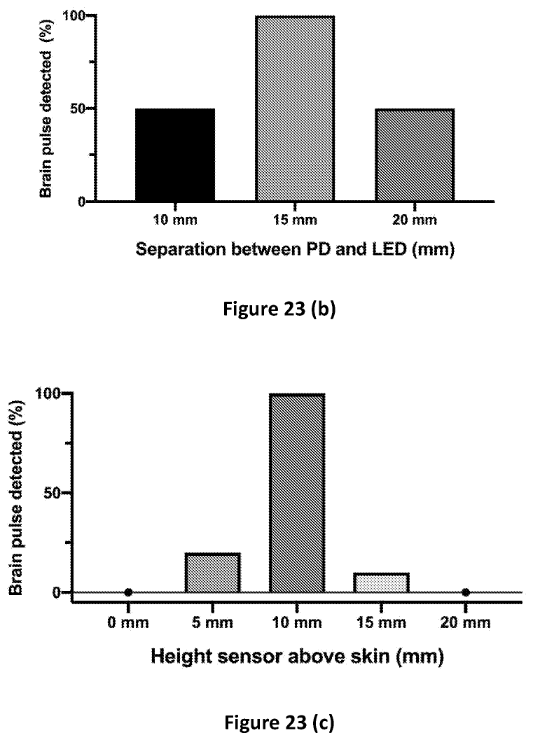

The spacing Y may be in the range of about 1 mm to about 20 mm. In some embodiments, the spacing Y may be in the range of about 7 mm to about 10 mm. The spacing Y may, for example, be about 8.5 mm. In a preferred embodiment of the invention the light source 120 (specifically the light emitting region of the light source 120) and photo-detector 130 (specifically the light sensitive region of the photo-detector 130) are set back from the contact surface 111 by about 7 mm to about 10 mm, such as about 8.5 mm, and centre points of the light source 120 and the photo-detector 130 are separated by from about 10 mm to about 20 mm, such as about 15 mm. In one aspect of this embodiment, the diameter of the light source 120 (or light emitting region) and the photo-detector 130 (or light sensitive region) is about 8 mm in each case. Experimental demonstration of optimal spacing between light emitting region and light sensitive region and of optimal spacing of the light emitting region and light sensitive region from the contact surface 11 is provided in Example 1 and FIGS. 23 (a) to (c).

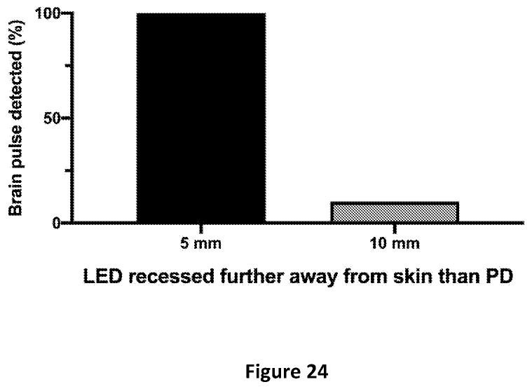

In other embodiments of the invention the light source 120 is spaced from the contact surface 111 up to 5 mm further than spacing of the photo-detector 130 from the contact surface 111. The efficacy of this embodiment is demonstrated in Example 1 and in FIG. 24.

The first and second recesses 112, 113 may have a depth D extending from the base 115 to a plane defined by the contact surface 111. The depth D may be in the range of about 1 mm to about 10 mm. The depth D may, for example, be about 8.5 mm. The depth D may be slightly less than the spacing Y (also referred to as “set back”) in the case where the light source 120 and the photo-detector 130 project into the recesses 112 and 113, respectively.

In some embodiments, the body 110 further comprises a wall 114 that separates the first recess 112 from the second recess 113. The wall 114 may extend from a base 115 of the body 110 towards the contact surface 111 to a wall height WH. The wall 114 assists in limiting emitted light from the light source 120 being reflected or scattered to the photo detector 130 without first interacting with the internal organ. The wall height WH of the wall may be at least about 2 mm. In some embodiments the wall height is about 4 mm. The wall height WH may be less than or equal to the depth D. The wall 114 may have a wall thickness WT in the range of about 1 mm to 2 mm. The wall thickness WT may, for example, be about 1.8 mm.

The light source 120 may be configured to emit light comprising at least two discrete wavelengths. For example, the first discrete wavelength may be centred around about 895 nm or about 940 nm or about 945 nm (longer wavelength, or a first wavelength) and the second discrete wavelength may be centred around about 660 nm (shorter wavelength or a second wavelength). The emitted light may comprise light with wavelengths in at least two discrete narrow bands of wavelengths.

In some embodiments, the shorter wavelength light may comprise light with wavelengths in the range of about 600 nm to about 750 nm. For example, the shorter wavelength may comprise wavelengths centred around about 660 nm with a range between about 640 nm and about 680 nm. The longer wavelength light may comprise light with wavelengths in the range of about 850 nm to about 1000 nm. For example, the longer wavelength may comprise wavelengths centred around about 895 nm with a range between about 855 nm and about 945 nm. In some embodiments, the longer wavelength is around 940 nm. The shorter wavelength light is absorbed more than longer wavelength light by blood with a low oxygen saturation (or low blood oxygen level). The longer wavelength light is absorbed more than shorter wavelength light by blood with a high oxygen saturation (or high blood oxygen level). As a result, different intensities at each wavelength band are reflected by the internal organ to be received and detected by the photo-detector 130. This principle is exploited to determine the blood oxygen levels and is explained in further detail below.

Light in the range of about 640 nm to about 680 nm is relatively sensitive to changes in the blood oxygen levels and is absorbed to a greater extent by deoxygenated blood than oxygenated blood. Accordingly, light reflected from the internal organ (or blood vessels associated with the internal organ) at these wavelengths is affected by the blood oxygen levels and the received light intensity may be used (in combination with light at a longer wavelength) to determine the blood oxygen level.

Light in the range of about 850 nm to about 1000 nm is relatively sensitive to changes in the blood oxygen levels and is absorbed to a greater extent by oxygenated blood than deoxygenated blood. Accordingly, light reflected from the internal organ (or blood vessels associated with the internal organ) at these wavelengths is affected by the blood oxygen levels and the received light intensity may be used (in combination with light at a shorter wavelengths) to determine the blood oxygen level.

Light in the range of about 780 nm to about 820 nm is relatively insensitive to changes in the blood oxygen levels and is absorbed to the same extent regardless of oxygen saturation levels. Accordingly, light reflected from the internal organ at this wavelength may therefore provide a more reliable signal from which to determine stages of the cardiac cycle and/or to make health assessments based on pulsatile changes in blood flow.

In some embodiments, the light source 120 is configured to emit light with wavelengths in a narrow middle wavelength band in the range of about 780 nm to about 820 nm. The light source 120 may be configured to emit light with wavelengths centred around about 805 nm. The amount of light in the middle wavelength band that is absorbed by the blood but is insensitive to the oxygen saturation of the blood. For example, the light source 120 may comprise a third LED adapted to emit light at the narrow middle wavelength band.

The light source 120 may comprise one or more semiconductor diodes such as a light emitting diode. The photo-detector 130 may also comprise one or more semiconductor diodes. The light source 120 and photo-detector 130 may be generally shaped as a short cylinder, e.g. a pill-shape. The light source 120 and photo-detector 130 may have a diameter Z of about 8 mm.

The light source 120 may have an optical power output of up to about 20 milliWatts (mW). In some embodiments, the light source 120 may comprise two LEDs with a total optical power output from both LEDs of up to about 20 mW, such as for example 50 microWatts (μW) to about 20 mW, about 100 μW to about 10 mW or about 200 μW to about 5 mW. In some embodiments, the light source 120 may comprise two LEDs with a total optical power output from both LEDs of about 100 μW, 200 μW, 500 μW, 10 mW, 15 mW, 20 mW or more than 20 mW.

The photo-detector 130 may be configured to detect a broad range of wavelengths. The light source 120 may be configured to produce pulsed light at sequentially different frequencies so that at any given time only light over a narrow bandwidth is emitted. A wavelength of light can then be associated with light detected by the photo-detector 130 based on the timing of detection.

In some embodiments, the photo-detector 130 is configured to detect discrete narrow band ranges of wavelengths corresponding to the wavelengths of emitted light. The photo-detector 130 may output a plurality of signals indicative of the intensity of light detected at each discrete narrow band range of wavelengths. In some embodiments, the photo-detector 130 may output a multiplexed signal of the plurality of signals.

The photo-detector 130 may be configured to generate one or more signals indicative of the intensity of light detected by the photo-detector 130. The apparatus 100 may be further configured to transmit the signals to a processor 562 (FIG. 5). The apparatus 100 may comprise a conductive cable 140 (or wire) to transmit the signal to a processor 562 or may wirelessly transmit the signals to the processor 562. The signals are indicative of the blood oxygen level of the internal organ.

The photo-detector 130 may, for example, be similar to the PIN photo-detector used in the Nellcor™ Maxfast forehead sensor by Medtronic.

In some embodiments, the apparatus 100 comprises at least two optical waveguides (e.g. optical fibres). The light emitting region of the light source 120 may therefore comprise an end of a first optical waveguide (not shown) located in the first recess 112. The photo-detector 130 may comprise a second optical waveguide (not shown) where one end is located in the second recess 113 and the light sensitive region may be located external to the body 110 of the apparatus 100.

Referring to FIGS. 3 and 4, the body 110 of the apparatus 100 may be formed from a plurality of components comprising a base 115 and spacer 116.

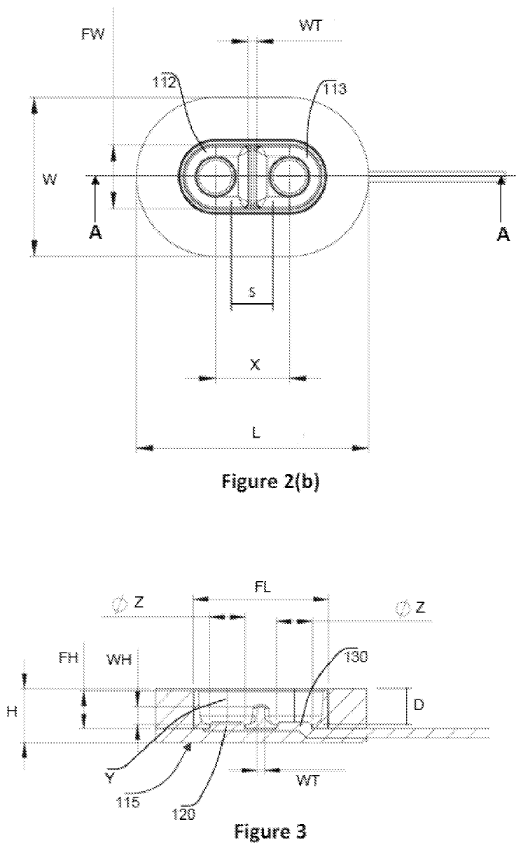

The base 115 may be formed from a rigid material, for example, a polymer such as ABS. The base 115 may define a recess 418 to accommodate at least part of the light source 120 and the photo-detector 130. The base 115 may have a base thickness BT of around 3 mm (see. FIG. 2a).

The spacer 116 may be formed from a soft foam. The spacer 116 may have a spacer thickness ST of around 9 mm (see. FIG. 2a). The body 110 may therefore have a total height H of about 22 mm. The spacer 116 may be used to define the spacing Y between the light emitting region from the subject 520 when the contact surface 111 engages the subject 520. Therefore, the spacer thickness ST may be greater than or equal to the spacing Y. The spacer thickness ST may be in the range of about 8 mm to about 11 mm. The spacer 116 and body 110 may have a length L of about 47 mm and a width W of about 32 mm.

In some embodiments, the body 110 further comprises a frame 450 configured to reside in a cavity 419 of the spacer 116. The cavity 419 extending from the contact surface 111 through the entire spacer thickness ST of the spacer 116.

The frame 450 may define the first recess 112, the second recess 113 and comprise the wall 114. The frame 450 may have a frame height FH extending from the base 115 towards the contact surface 111. The frame height FH may be less than the spacer thickness ST and an upper rim 451 of frame 450 may not reach the plane defined by the contact surface 111 when the frame 450 is located within the cavity 419. The frame height FH may be less than about 21 mm. In some embodiments, the frame height FH may be less than about 11 mm. The frame height FH may be about 8.5 mm. The frame 450 may have a frame length FL of about 30 mm and a frame width of about 13 mm. The frame 450 may be formed from a rigid material, for example, a polymer such as ABS.

The frame 450 may define a first aperture 452 to enable light from the light source 120 to be emitted out of the first recess 112 and a second aperture 453 to enable light from the second recess 113 to be received by the photo-detector 130. In some embodiments, the first aperture 452 and the second aperture 453 are configured to allow upper portions (light emitting region) 432, (light sensitive region) 433 of the light source 120 and the photo-detector 130, respectively, to protrude into the respective first and second recesses 112, 113.

In some embodiments, the light source 120 and photo-detector 130 are commonly housed on a support 440. The cavity 418 of the base 115 may be configured to receive the support 440.

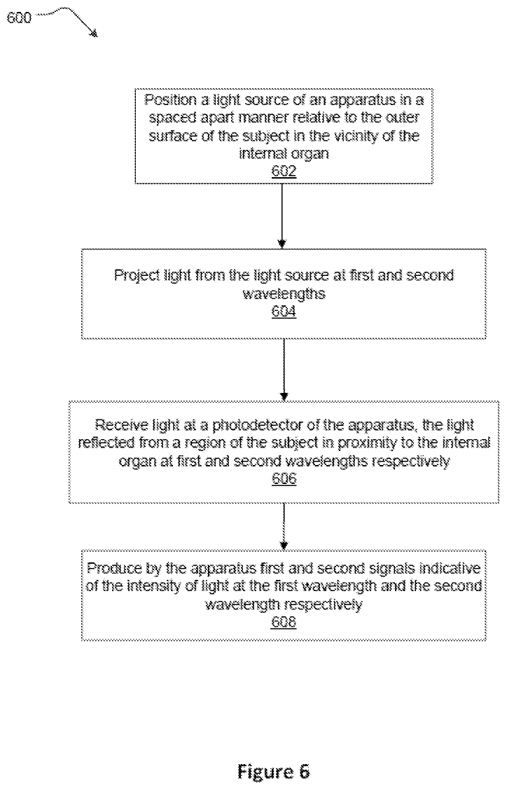

Referring to FIG. 6, a process flow diagram for a method 600 of obtaining data indicative of blood oxygen levels of an internal organ of a subject 520 is shown.

The method 600 comprises positioning an apparatus 100, 550 from an outer surface (e.g. skin) of the subject 520 in a vicinity of or adjacent the internal organ such that a light source 120 of the apparatus 100, 550 is spaced apart from the outer surface, at 602.

The apparatus projects light from the light source 120 through the outer surface to the internal organ, at 604. The light comprises at least a first and second wavelength of light.

The photo-detector 130 of the apparatus 100 receives light at the first and second wavelength after the projected light has interacted with the internal organ. For example, the received light may have been partially absorbed and reflected by the internal organ.

The apparatus 100 produces a first signal indicative of the intensity of light at the first wavelength and a second signal indicative of the intensity of light at the second wavelength, at 608. For example, the first wavelength may be about 660 nm and the second wavelength may be about 895 nm. In some embodiments, the apparatus 100 also produces a third signal indicative of the intensity of light at a third wavelength. For example, the third wavelength may be in the range of about 780 nm to about 820 nm, or about 805 nm.

In some embodiments, a first apparatus 100 is located on the subject in the vicinity of the internal organ to target the internal organ of the subject. This enables the light produced by the light source 120 of the apparatus 100 to be projected through the outer surface of the subject to a region of the internal organ. The light produced may, for example, interact with the microvasculature and veins of organs of the body. The microvasculature of organs of the body may, for example, comprise any one or more of: arterioles, capillaries and venules. The light received by the photo-detector 130 may therefore be representative of the blood oxygen level of the microvasculature or veins of the organs. This may be used to assess the tissue oxygen levels of the organ, as the low oxygen levels reached in the venules and veins are in equilibrium with extravascular tissue oxygen levels.

However, the projected light may also interact with the skin of the subject which also comprises blood vessels. This may affect the light received by the apparatus 100 and therefore influence the signals produced. It was found by the inventor that by spacing the light source 120 from the outer surface (e.g. skin) of the subject 520, so that the light source 120 and photo-detector 130 do not touch the skin, the light received by the photo-detector 130 is dominated by light that indicative of blood oxygen levels in the internal organ. Moreover, the received light has minimal contributions from light that is indicative of blood oxygen levels arising from the skin of the subject. The intensity of the light source 120 may be optimised to minimise the contribution from light reflected from the skin.

### Positioning of Apparatus to Target Brain

In some embodiments, where the internal organ being assessed is the brain, the apparatus 100 may be located on the scalp at a location where the skull is relatively thin compared to other locations of the skull. For example, suitable locations may include the temples, occipital, orbital, parietal and frontal areas of the skull.

Due to the novel design of the apparatus, which has a relatively small separation distance between the photodetector and LED, the apparatus (or sensor head) may be configured, such as modified and miniaturized, to allow placement in the right or left ear canals. Placement in the ear canals allows for the health of the temporal lobe and cerebellum of the brain, which are adjacent to the ear canal, to be assessed. The apparatus 100 may also be inserted in the ear canals.

In some embodiments, the apparatus 100 is located at a position over the sulcus of the brain 521 of the subject 520, such as the lateral sulcus (Sylvian fissure). Placing the apparatus 100 at or over the lateral sulcus or other cortical sulcuses may lead to the projected light interacting with cerebrospinal fluid (CSF) covering the brain. In such a situation, the signals derived from the detected light may be dominated by the effects of movement of the CSF in response to the pulsatile changes in intra-cranial pressure, as each arterial pressure pulse of blood into the skull causes a pulsatile change in intra-cranial pressure levels. As discussed in more detail below, signals derived from the apparatus when it is placed at or over the lateral sulcus or other cortical sulcuses of a subject may be used to determine and/or monitor intra-cranial pressure changes in the brain.

### Positioning of Apparatus to Target Lung

In some embodiments, the apparatus 100 may be arranged to be located on the sternal notch, on supra-clavicular spaces, or between ribs of the subject 520 such that the apparatus 100 is near a lung of the subject 520 to obtain data indicative of blood oxygen levels of the lung.

### Positioning of Apparatus to Target Liver

The apparatus 100 may be arranged to be located below ribs in right upper quadrant or the epigastrium of the subject such that the apparatus 100 is near a liver of the subject 520 to obtain data indicative of blood oxygen levels of the liver.

### Positioning of Apparatus to Target Intestines

The apparatus 100 may be arranged to be located on the abdomen or either of the lower quadrants of the subject such that the apparatus 100 is near intestines of the subject 520 to obtain data indicative of blood oxygen levels of the intestines.

### Positioning of Apparatus to Target Kidney

The apparatus 100 may be arranged to be located on the back of a subject such that the apparatus 100 is near a kidney of the subject 520 to obtain data indicative of blood oxygen levels of the kidney.

Positioning of apparatus to target skeletal muscle The apparatus 100 may, for example, be arranged to be located over the muscle of a subject such as the leg muscle, to target skeletal muscle such as the gastrocnemius (or calf muscle), to obtain data indicative of blood oxygen levels of the skeletal muscle.

### Positioning of Apparatus to Target the Right Ventricle of the Heart

The apparatus 100 may, for example be arranged to be located over the sternum of the chest or along the left border of the sternum where it meets the ribs to target the right ventricle of the heart.

### Positioning of Apparatus to Target Foetus

The apparatus 100 may be arranged to be located on the abdomen of the subject 520 such that the apparatus 100 (transabdominal sensor) is near the uterus (womb) of the subject 520 to obtain data indicative of blood oxygen levels of an internal organ of a foetus within the uterus. The foetus has a different heart rate and rhythm and pulse shape compared with the mother. The foetus also has a different blood oxygen level compared to the mother.

In some embodiments, the apparatus 100 may comprise a light source adapted to be intra-vaginally located within a mother to enable light to be projected onto the brain of a foetus to enable measurement of brain oxygen levels during delivery of the foetus.

In some embodiments, multiple apparatus 100, 550 may be used to simultaneously or substantially simultaneously obtain data from the same internal organ. For example, this can be used to determine any differences between different regions of the internal organ or obtain additional data to enable a more accurate result to be obtained that is more representative of the entire organ. In some embodiments, two apparatus 100 are placed on the scalp, one on either side of the head, to obtain data from the skin's arterial pulse and also for each hemisphere of the brain 521.

The apparatus 100 may be configured to determine data simultaneously from multiple organs or sites of the body and also the skin. This may be useful in detecting systemic and regional disorders of the body by monitoring abnormal combinations of patterns of blood oxygen levels, pulse shape, pulse amplitude, and/or organ movement. This allows a determination of whether changes in blood flow or oxygen levels are systemic (occurring at multiple sites) or regional (occurring at just one site).

Referring to FIG. 5, a system 500 for assessing health of the subject 520 is shown, according to some embodiments. The system 500 comprises a processor 562, memory 568 coupled to the processor 562. The system 500 may further comprise an apparatus, such as apparatus 100, for determining data indicative of blood oxygen levels of internal organs. The system 500 may comprise a computing device 560 including the processor 562, a display 564 and a user interface 566. The processor 562 may be configured to execute instructions (computer readable instructions or code) stored in memory 568 to performed described methods, such as assessing the health of the subject based on one or more signals indicative of blood oxygen levels of the internal organ received from the apparatus 100. For example, the apparatus 100 may be connected to the processor 562 via a conductive cable 140 or wirelessly.

In some embodiments, the system 500 may also comprise a second apparatus 550 for determining data indicative of blood oxygen levels. The second apparatus 550 comprises a second light source and a second photodetector configured to produce a further signal. For example, the further signal may be indicative of the timing and the characteristic waveform of an arterial pulse of the subject 520. The second photodetector is configured to transmit data representative of the further signal to the processor 562. In some embodiments, the processor 562 may be configured to receive a first and second signal from the first apparatus 100 and a third signal from the second apparatus 550.

The second light source may be adapted to produce light at the narrow medium wavelength band discussed above. The second photodetector may be adapted to receive and detect light at the narrow medium wavelength band accordingly.

In some embodiments, the second apparatus 550 may be a component of a conventional skin pulse oximeter. The second apparatus 550 may be configured to receive reflected light from the subject 520. Alternatively, the second apparatus 550 may be configured to be placed on a part of the subject such as a forehead, nose, ear or finger and receive light transmitted through the part of the subject 520.

The second apparatus 550 may be positioned on or proximate to the outer surface (e.g. skin) of the subject 520 at a separate location to the location of the first apparatus 100 such that the third signal is indicative of complementary information including pulse shape, pulse amplitude, relative timing of the pulse and blood oxygen levels relative to a separate location. This is discussed in further detail below.

In some embodiments, the computing device 560 comprises an analogue interface (not shown) connecting the apparatus 100, 550 to the processor 562. The analogue interface may comprise, for example, an ‘Integrated Analog Front-End for Pulse Oximeters’ model AFE4490 from Texas Instruments. The analogue interface may also provide electrical power to the apparatus 100, 550.

In some embodiments, the system 500 further comprises sensors attached to catheters and placed in the body of a subject, such as endotracheal tubes (assess lungs, pulmonary artery), nasogastric tubes (assess lung, heart, liver, oesophagus, stomach, duodenum), urinary catheter (assess bladder, intestine), cerebrospinal fluid ventricular drains (assess brain), routine post-operative drains in abdomen (assess liver, intestine), and/or chest tubes (assess heart and lungs). The processor 562 may be configured to receive and process signals received from the sensors to assist in making heath determinations about the subject.

In order to accurately assess the health of a subject based on received signals, the received one or more signals will preferably be predominantly (or dominated by) signals indicative of blood oxygen levels of the internal organ being targeted. For example, it is understood that poor placement of the apparatus relative to the internal organ may result in the one or more signals comprising contributions or information derived from light reflected by the skin of the subject as well as light reflected by the targeted internal organ.

By determining that a waveform associated with the signal is representative of a signal predominantly associated with the targeted internal organ 521 before proceeding to assess the health of the subject based on the signal, described embodiments provide for more accurate assessments of the health of the subject.

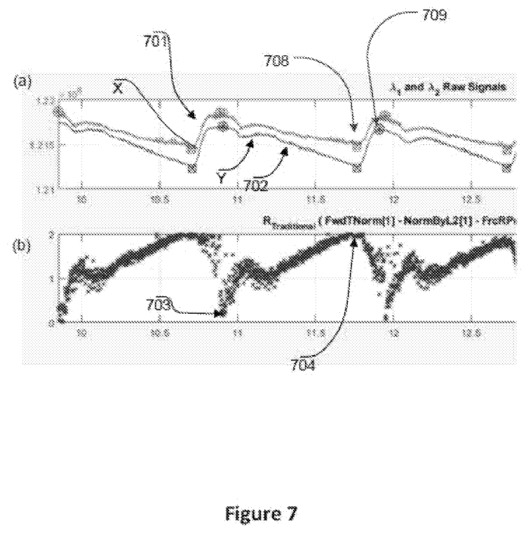

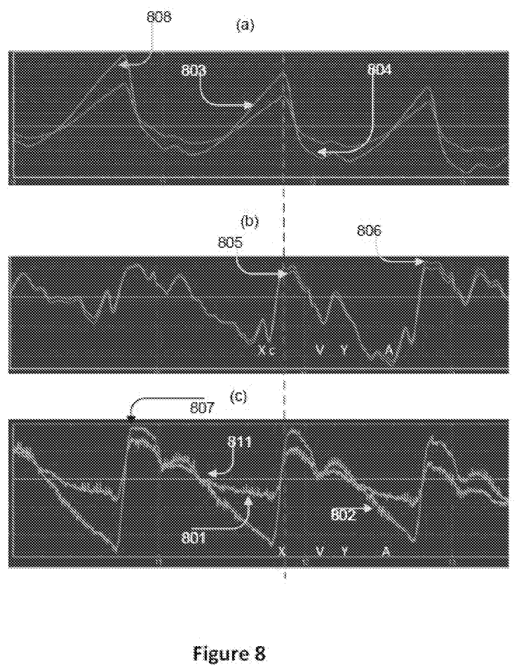

For some internal organs, a signal with venous circulation characteristics is expected and accordingly, the waveform may be compared with a typical venous signal, which has the characteristics of the waveform of central venous blood pressure trace or a measured venous waveform to determine that the signal is predominantly associated with the internal organ. This is explained further with reference to FIGS. 7 and 8, where waveforms associated with first and second signals arising from an internal organ are being analysed. However, it will be appreciated that health assessments of the internal organs may also be determined based on a waveform associated with a single signal.

Referring to FIG. 7(a), an example of a first waveform of a first signal 701 derived from measured light reflected from the internal organ at a first wavelength and a second waveform of a second signal 702 derived from measured light reflected from the internal organ at a second relatively shorter wavelength is shown. In this example, the first wavelength is around 895 nm and the second wavelength is around 660 nm. These first and second signals 701, 702 were obtained from an apparatus 100 placed on the subject 520 in the vicinity of the human brain 521. The amplitude or level of the signals is representative of the intensity of light detected by the photo-detector 130 and plotted as a function of time.

Blood oxygen levels in the microcirculation of all regions of the body, other than the lungs, typically increase during the systole stage of a cardiac cycle and fall during the diastole stage, as oxygen moves from the blood and into the tissues. This leads to respective changes in the signal levels detected. The first waveform and the second waveform of the respective first signal 701 and second signal 702 show a plurality of peaks and troughs representative of intensity levels over time and are indicative of the pulse of the subject. The signals 701, 702 may be described as pulsatile signals and/or plethysmographic signals.

In some embodiments, a second conventional apparatus 550 may be located at a separate location to the first apparatus 100 to produce a third and fourth signal indicative of blood oxygen levels in the skin. The separate location may be, for example, any one of: a forehead, a finger, an ear, and a nose, of the subject 520. The third and/or fourth signals may be indicative of pulse shape, relative timing of the pulse and the arterial blood oxygen levels from the separate location.

FIG. 8(a) shows an example of a third waveform of a third signal 803 derived from measured light reflected from the skin at a third wavelength and a fourth waveform of a fourth signal 804 derived from measured light reflected from the skin at a fourth relatively shorter wavelength produced by the second apparatus 550. In this example, the third wavelength is around 895 nm and the fourth wavelength is around 660 nm. The third and fourth waveforms of the respective third and fourth signals 803, 804 show a plurality of peaks and troughs representative of intensity levels over time. The third and fourth signals 803, 804 were obtained from the forehead of a human subject 520 and are representative of a skin pulsatile arterial signal. That is, the third and fourth waveforms are characteristic of the pressure waveform of an arterial circulation signal in the skin.

FIG. 8(b) shows an example of a simultaneous fifth waveform of a fifth signal 805 derived from measured light reflected from the internal jugular vein at a fifth wavelength and a sixth waveform of a sixth signal 806 derived from measured light reflected from the internal jugular vein at a sixth relatively shorter wavelength produced by the second apparatus 550. In this example, the fifth wavelength is around 895 nm and the sixth wavelength is around 660 nm. The fifth and sixth waveforms of the respective fifth and sixth signals 805, 806 show a plurality of peaks and troughs representative of intensity level over time. The fifth and sixth signals 805, 806 with the second apparatus 550 placed over an internal jugular vein of the subject 520 and are therefore representative of a pulsatile venous circulation signal. The fifth and sixth signals 805, 806 shown are representative of the shape of the venous blood pressure changes that are typically observed in a large vein when pressure levels are monitored. That is, the fifth and sixth waveforms are characteristic of a venous circulation pressure signal.

Similar to the plot of FIG. 7(a), FIG. 8(c) depicts a first waveform of a first signal 801 derived from measured light reflected from the internal organ at a first wavelength and a second waveform of a second signal 802 derived from measured light reflected from the internal organ at a second relatively shorter wavelength. These first and second signals 801, 802 were obtained from sensors of an apparatus 100 placed on the subject 520 in the vicinity of the brain 521 at the same time as the detected light for the plot of FIG. 8(a). The amplitude or level of the signals 801, 802 is representative of the intensity of light detected by the photo-detector 130 and plotted as a function of time. It can be seen from FIG. 8(c) that the shape of the first and second waveforms of the first and second signals 801, 802 obtained from the brain using apparatus 100 are a better match for the waveform shape of a venous signal (as depicted in FIG. 8(b)) than an arterial signal (as depicted in FIG. 8(a)). Thus, it can be deduced that the first and second signals 801, 802 are likely to have been derived from received light that has predominately interacted with the surface of an internal organ, in this case the brain 524, where the majority of blood resides in venules and veins. The brain signal is therefore unlike the skin signal waveform and can be used to determine that the signal is arising from the brain and not from the skin.

Referring again to FIGS. 8(b) and 8(c), components of the waveforms may correspond with features typically found in venous blood pressure waveforms from veins such as A, C, X, V and Y waves. For example, the first and/or second waveforms may comprise A, C, X, V and/or Y wave components corresponding to A, C, X, V and Y waves typically observed in the pressure signal from a vein (venous signal).

The A-wave component may be observed as a large trough in the signal value of the waveform. The A-wave typically occurs and the end of the diastolic stage of a cardiac cycle due to atrial contraction. The C-wave component may be observed as a small trough superimposed over the signal value of the waveform after the minimum signal value of the A-wave. This typically occurs at the beginning of the systolic stage of the cardiac cycle due to tricuspid valve bulging. The X-wave component may be observed as an increase in the signal value after the minimum point in the A-wave. The X-wave typically begins at the end of the diastolic stage of the cardiac cycle as blood is emptied from the heart and therefore occurs during the systolic stage. The V-wave component may be observed as a trough superimposed over the signal value of the waveform after the X-wave and the peak signal value. The V-wave typically occurs during the late systolic stage of the cardiac cycle due to filling of the atrium of the heart. The Y-wave component may be observed as an increase in the signal value after local maximum value of the V-wave. The Y-wave typically occurs during the early diastolic stage of the cardiac cycle as the ventricle of the heart begins to fill.

In some embodiments, determining that the waveforms are representative of signals predominantly associated with the targeted internal organ comprises determining that the waveform comprises at least one of an X-wave component, an A-wave component, a C-wave component, a V-wave component and an Y-wave component.

As is shown in later figures, signals indicative of light reflected from an internal organ, such as brain, lung, liver, intestine and also foetal organs, tend to exhibit characteristics of a venous signal at least at one wavelength. For skeletal muscle and heart signals, typical venous features may not be present.

This knowledge may be used to analyse one or more signals from the apparatus 100 to determine or confirm that at least one of the waveforms of the signals is representative of a signal associated with the internal organ of the subject 520. For example, in some embodiments, determining that the waveforms of the one or more signal received from the apparatus 100 is representative of a waveform of a signal associated with the internal organ of the subject 520 may comprise determining if the waveforms is representative of waveforms of venous signals.

In some embodiments, a further waveform from a further signal indicative of the pulse of the subject, such as signal 803 depicted in FIG. 8(a), may be compared with the one or more waveforms to confirm that at least one of the waveforms of the signals is representative of signals associated with the internal organ of the subject 520. For example, it is expected that the waveform would depict a component of the signal, such as the start of the brain pulse (maximum signal intensity) that lags in time compared with a corresponding component of the skin pulse. This reflects the time for the blood to move through the microcirculation and reach the brain venules. In some embodiments, the further or third signal may be derived from reflected light with a wavelength in the range of about 780 nm to about 820 nm. In some embodiments, derived from light reflected from the skin of the subject at a wavelength of any one of: around 660 nm, around 805 nm, around 895 nm or around 940 nm.

The one or more signals arising from or predominantly associated with different internal organs may each have a characteristic waveform. The characteristic waveforms may be different for different internal organs, as depicted in FIGS. 7, 8, 10 to 16 and 20 and discussed in more detail below. A waveform from at least one of the one or more signals may be compared to one or more of the characteristic waveforms to determine if the waveform is sufficiently similar to any one of the characteristic waveforms. The comparison may be used to determine if the one or more signals are representative of signals associated with the internal organ and/or to determine the health condition of the subject 520 as will be discussed in more detail below. In any case, in some embodiments, if the comparison is used to determine if the one or more signals are representative of signals associated with the internal organ as well as to determine the health condition of the subject 520, different tolerances or thresholds for similarity may be applied. For example, a lower threshold for similarity may be applied when assessing whether the one or more signals are representative of signals associated with the internal organ and a relatively higher threshold for similarity may be applied when determine the health condition of the subject 520.

In some embodiments, a modified ratio of ratios may be calculated from the signal levels of two or more signals over the respective waveform. The modified ratio of ratios value is indicative of the blood oxygen level and may be used to determine if the two or more signals are representative of signals associated with the internal organ or to determine the health condition of the subject 520. This is discussed in more detail below.

In order to compare a two or more waveforms, such as the waveform associated with the signal from the internal organ and a characteristic waveform, it is preferably that the same portion or window of pulsatile signals are being compared. With reference to FIG. 7 for illustrative purposes only, in some embodiments, the start of the window of the waveform may be determined as at the time of a peak signal level 707 of the second signal 702 and the end of the window of the waveform may be determined as at the time of the minimum signal level 708 of the second signal 702. In some embodiments, the end of the window of the waveform may be determined as at the time of the subsequent peak signal level 709 of the second signal 702. The window of the waveform may comprise a time-limited section of the signals 701, 702, 801, 802, 803, 804. For example, the window of the waveform may comprise the section of the signals 701, 702, 801, 802, 803, 804 from a first extrema of the signal level to a second extrema of the signal level (or to immediately prior to the second extrema). The window may, for example, comprise the section of the signals 701, 702, 801, 802, 803, 804 from a first peak level to a second peak level (or to immediately prior to the second peak level). The window may therefore begin at the leading edge of the A-wave and end at the end of the X-wave. In some embodiments, the window of the waveform may, for example, comprise the section of the signals 701, 702, 801, 802, 803, 804 from a first minimum signal level to a second minimum signal level (or to immediately prior to the second minimum signal level). The window waveform may therefore begin at the X-wave and end at the trough of the A-wave.

In some embodiments, determination of the start and end of the window for comparing the waveforms of the signals may be determined using the waveform (or signal levels) of a further signal, such as an arterial signal 803, 804 or a jugular vein signal 805, 806. The window of the waveform may, for example, be determined to start at a time of a peak signal level of the separate signal and end at the time of the minimum signal level of the separate signal. The separate signal may be derived from light at a wavelength in the range of about 780 nm to about 820 nm.

In some embodiments, analysis of the waveform from the further signal may also be used to determine the timing of the systolic and diastolic stages of the cardiac cycle. This may be useful where it is not particular easy to determine the stages of the cardiac cycle from the waveforms of the one or more signals. For example, it may not be straightforward to determine the A-wave and X-wave components from the waveforms of the one or more signals to thereby determine when the systolic and diastolic stages occur.

The waveforms may comprise a discrete part of the respective signal over a specific time period. In some embodiments, the waveform may comprise a portion of a wavelength of the signal indicative of only a portion of the pulse of the subject, may comprise a wavelength of the signal indicative of a single pulse of the subject or may comprise a plurality of wavelengths of the signal indicative of a plurality of pulses. Once a window or waveform has been determined, an average or summed waveform may be generated from a plurality of waveforms to improve the signal-to-noise ratio. In some embodiments, a window function may be applied to the first and/or second signals to derive the respective first and second waveforms. For example, the window function may comprise a rectangular, triangular, smoothing and/or bell-shaped curve function.

In some embodiments, the waveforms of the one or more signals is selected based on the waveform (or signal levels) of a further signal derived from light at a third wavelength in the range of about 780 nm to about 820 nm. The waveform may, for example, start at the time of a peak signal level of the third signal to a minimum signal level of the further signal. Light having wavelengths in the third wavelength range is sensitive to blood but insensitive to changes in the blood oxygen levels and may therefore provide a more reliable signal to determine stages of the cardiac cycle from and the shape of the waveform arising from pulsatile blood flow. This is particularly useful for determining the waveform for complex signals such as those obtained from the lungs and sometimes the brain. For example, a further signal based on light at a wavelength of about 805 nm may be used to recognise disorders of microvascular blood flow to an organ based on the waveform (or pulse shape and amplitude) of the further signal. The further signal may also be used for the templates discussed elsewhere to define the characteristic waveforms of organs to determine that the signal is representative of a given organ.

Although FIGS. 7, 8 and 10 to 16 and 20 are shown in the time domain, it will be appreciated that the waveforms may be analysed in the time domain or the frequency domain.

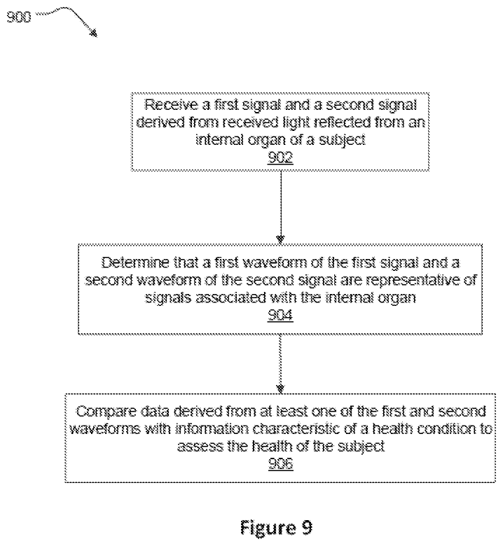

Referring now to FIG. 9, a process flow diagram for a computer-implemented method 900 of assessing the health of a subject 520, according to some embodiments, is shown. The method 900 may be implemented by the processor 562 executing instructions stored in memory 568.

The processor 562 receives one or more signals, at 902. The one or more signals are derived from received light reflected from a region 522 of a subject 520 in proximity to an internal organ 521 at first and second wavelengths respectively. For example, the received light may comprise the emitted light that has interacted with the internal organ 521.

The processor 562 determines that at least one of one or more respective waveforms of the one or more signals is representative of signals predominantly associated with the internal organ 521, at 904. The determination of whether the one or more waveforms is representative of a signal associated with the internal organ is discussed in more detail below.

In some embodiments, in response to the processor 562 not determining that at least one of the one or more respective waveforms of the one or more signals is representative of a signal associated with the internal organ, the processor 562 may output an error or control signal. For example, the control signal may be provided to the apparatus to cause or instruct the light source 120 of the apparatus 100 to be repositioned relative to the outer surface of the subject 520. In some embodiments, the apparatus may be configured to automatically reposition the light source 120. In some embodiments, the system may be configured to instruct an operator to reposition the apparatus relative to the internal organ being targeted. Once repositioned, updated one or more signals may be provided to the processor 562 for processing.

The processor 562 compares data derived from at least one of the one or more waveforms with information characteristic of a health condition to assess the health of the subject 520, at 906. The processor 562 may analyse at least one of the one or more waveforms to derive the data, which may, for example, comprise de-convoluted components of the waveform or calculated gradients (or rates of change).

In some embodiments, the processor 562 may assess the health of the subject 520 and output an assessment of the health of the subject 520. For example, the assessment may be output to the display 564 or to any other user interface devices, such as a speaker or a third party device. Responsive to determining that the subject 520 is likely to have a health condition, the processor 562 may send a signal to cause information based on the assessment to be outputted via the display 564 and/or a speaker and/or alarm) to be shown on the display 564 and/or cause an audible alert to sound. In some embodiments, a control signal may be transmitted to monitoring or controlling equipment coupled to the subject to adjust settings of the equipment. Determination of whether the subject 520 is likely to have a health condition is discussed in more detail below.

In some embodiments, the processor 562 may be configured to display one or more of: oxygen levels throughout the whole of the systolic and diastolic stages of each cardiac cycle, the estimated tissue oxygen level of the organ, (based on the trough level reached during the diastolic phase), the respiratory inspiratory and expiratory oscillations in oxygen levels, the skin and organ plethysmography signals used to derive oxygen levels and the organ and skin plethysmography signal arising from 805 nm, which may be used to recognise disorders of microvascular blood flow in an organ, organ movement (brain, liver, lung and heart) associated with the respiratory and cardiac cycles, any asymmetry between the signals from the left and right hemispheres of the brain, or other combinations of sensors across multiple organs of the body.

As previously mentioned, the inventor has recognised that waveforms associated with signals that are predominantly arising from or associated with internal organs exhibit specific characteristics, and are generally significantly different from waveforms associated with signals that are predominantly arising from or associated with skin. In some embodiments, the processor 562 is configured to create or generate the one or more templates for specific internal organs based on one or more signals received from apparatus 100 when apparatus 100 is arranged to target the specific internal organ. In some cases, an operator may arrange the apparatus 100 relative to the specific internal organ and analyse a waveform resulting from the received signal to verify that the signal is representative of the internal organ. Multiple signals derived from one or more subjects may be used to create the templates for specific internal organs based on the determined characteristic waveforms for the specific internal organs. Accordingly, the one or more templates stored in memory 568 may be based on a library or database of characteristic waveforms previously obtained from specific internal organs. The database may comprise a plurality of characteristic waveforms, each characteristic waveform being associated with a particular internal organ. For example, the database may comprise characteristic waveforms for any one or more of: brain, foetal brain, lung, liver, kidney, intestine, skeletal muscle, heart, and foetal heart. Each characteristic waveform may be based on a waveform from one or more previously received signal from the internal organ that the characteristic waveform is associated with. For example, one or more characteristic waveforms may comprise an average or sum of a plurality of previously received signals from same type of internal organ from different subjects. In some embodiments, one or more characteristic waveforms may be based on a theoretically expected (or an idealised) waveform for the internal organ. In some embodiments, the one or more templates may comprise non internal organ specific waveforms, such as waveforms associated with signals that are predominantly arising from or associated with skin.

In some embodiments, the processor 562 may receive information indicative of the internal organ being targeted with apparatus 100. The processor 562 may use the information indicative of the internal organ being targeted to assist in the determination that the one or more waveforms are representative of a signal predominantly associated with the target internal organ. The processor 562 may also use the information indicative of the internal organ being targeted to assist in the determination of the health condition. For example, the processor 562 may use the information on the type of internal organ to select a template corresponding to the relevant target internal organ, and thereby reducing processing time. In some embodiments, the processor 562 may therefore only compare the one or more waveforms with templates associated with the known targeted internal organ.

Determine if Waveform is Representative of a Signal Predominantly Associated with the Internal Organ

In some embodiments, memory 568 comprises one or more templates, each template comprising information characteristic of a particular internal organ, and in some embodiments, characteristic of a typical or healthy internal organ. For example, the templates may depict a characteristic waveform of the light intensity against time. The templates may comprise a characteristic plot of the modified ratio of ratios or blood oxygen levels. The processor 562 may be configured to compare at least one or more waveforms with the one or more template waveforms to determine if it is representative of a signal predominantly associated with the internal organ 521. The comparison may, for example, comprise calculating a difference between the waveform and the template waveform and comparing it with a threshold to determine a likelihood of the signal being predominantly associated with the internal organ. The difference between a plurality of template waveforms may be calculated to determine a best fit template waveform. This determination may comprise, for example, calculating a least sum of squared residual fit. If the sum of squares residual is less than a threshold error value, the processor 562 may determine that the waveforms is representative of (or a good match for) a signal being predominantly associated with the internal organ 521.

In some embodiments, the one or more waveforms may be compared with a typical venous waveform and responsive to the processor determining that at least one of the one or more signals substantially corresponds with the typical venous waveform, determining that the at least one of the one or more signals is dominated by a signal derived from the internal organ. For example, and as discussed above, an venous waveform typically comprises A, C, X, V and/or Y wave components.

In some embodiments, a further or third waveform may be derived from a further signal obtained from a second apparatus 550. The further waveform may, for example, be representative of an arterial skin pulse obtained from a location away from or peripheral to the internal organ. The further waveform may be used to compare to the one or more waveforms in terms of shape, amplitude and timing to determine if at least one of the first waveform and the second waveform is representative of an arterial pulse and therefore not a signal associated with the internal organ 521, but more likely arising from the skin.

In some embodiments, the processor 562 may receive an arterial signal, such as signal 803 depicted in FIG. 8(a), from sensors of the apparatus placed on the skin of the subject to obtain a further arterial waveform indicative of a skin arterial pulse of the subject 520. The processor 562 may compare the further waveform to the one or more waveforms derived from an internal organ of the subject 520 to determine whether a signal peak 807 of at least one of the one or more waveforms is offset in time from a respective signal peak 808 of the further waveform. For example, the one or more waveforms may depict a component of the signal, such as the peak signal level lags a corresponding component of the further signal in the further waveform (the peak signal level represents the start of the waveform).

In some embodiments, where the one or more signals comprises at least a first signal associated with a first waveform and a second signal associated with a second waveform, a modified ratio of ratios may be used to determine if the first and second signals are representative of signals associated with the internal organ or to determine the health condition of the subject 520. This is discussed in more detail below.

### Signal Waveforms—Brain

If the internal organ 521 is a brain, one or more signals indicative of light at different wavelengths and associated with the brain are expected to be similar to a venous signal. Therefore, in some embodiments, determining that at least one of the waveforms is representative of signals predominantly associated with the internal organ comprises determining that that at least one of the waveforms corresponds to a waveform of a venous pulse. In other embodiments, determining that at least one of the waveforms is representative of signals predominantly associated with the internal organ comprises determining that that at least one of the waveforms does not correspond to a waveform of an arterial pulse.

In some embodiments, the one or more signals comprise a first signal and a second signal. The first wavelength of the light from which the first signal is derived may be shorter than the second wavelength of light from which the second signal is derived. For example, the second wavelength may be about 660 nm. Where the internal organ being targeted is the brain, the second signal 702, 802 derived from light reflected by the brain at the second wavelength of about 660 nm may be more consistently representative of a venous signal. Accordingly, in some embodiments, the processor 562 uses the second signal 702, 802 to determine if the waveform is representative of a signal predominantly associated with the brain. In some embodiments, the first wavelength is about 805 nm. Changes in calculated oxygen levels or modified ratio of ratios could also be used as a template for this purpose and this is discussed below.

In some embodiments, the processor 562 is configured to analyse the one or more waveforms to determine one or more components to be compared to one or more corresponding components of one or more template waveforms. For example, the processor 562 may be configured to determine that a pulse shape of at least one of the one or more waveforms corresponds substantially with a characteristic brain pulse shape. The characteristic brain pulse shape may be defined by a template waveform such as a reference curve.

The characteristic brain pulse shape may comprise a first component with a generally increasing signal level (corresponding to the X-wave) at a first rate followed by a second component with a generally decreasing signal level (corresponding to the leading edge of the A-wave) at a second rate that has a smaller magnitude than the first rate of the X-wave. Accordingly, the processor 562 may determine the gradient or rate of change of the first and second components.

In some embodiments, the processor 562 may analyse the one or more waveforms to de-convolute the components from the waveform(s). The processor 562 may analyse the components to determine the gradient or rate of change and compare with a characteristic gradient or rate of change to determine if at least one of the one or more signals are representative of signals derived from the internal organ (same approach also applies to change in oxygen levels over the pulse duration).

It is expected that a pulsative signal representative of an internal organ may be offset in time from a third signal representative of an arterial signal such as obtained from the skin of a subject 520 (e.g. from the forehead, nose, ear). Therefore, responsive to determining that a signal peak 807 of at least one of the respective waveforms of the one or more signals is offset in time from a respective signal peak 808 of a further waveform of a further signal, the processor 562 may determine that at least one of the respective one or more signals is representative of the brain.

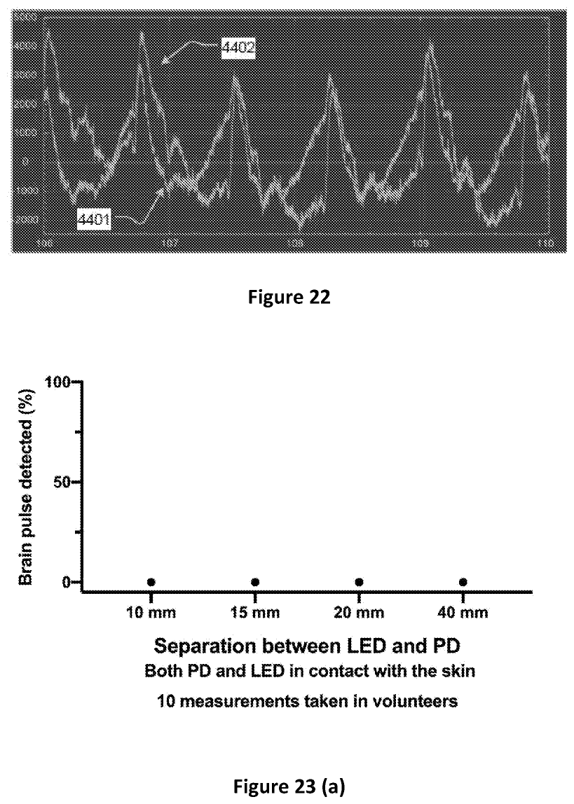

FIG. 22 shows a first signal 4401 (about 895 nm) and a second signal 4402 (about 660 nm) obtained from the apparatus 100 located in the ear canal of a human subject. In this instance, the processor 562 may be configured to determine that at least one of the waveforms is representative of signals predominantly associated with the brain by analysing the second signal 4402.

### Signal Waveforms—Lungs

The signal derived from light reflected by the lungs has been found to be relatively complex and may depend on a number of additional factors. These factors may include whether the alveoli (air sacs) are ventilated and the blood flow (perfusion) to the air sacs.

The blood flow may be dependent on the postural position of the person. Unlike other organs of the body, oxygen levels fall during systole and increase during diastole. In addition, the changes in oxygen levels during the pulse period are very large.

In some embodiments, in order to obtain one or more signals predominantly associated with the lung, the apparatus 100 may be positioned in the vicinity of the upper region of the lungs (e.g. near the apex of the lungs) where the lungs are above heart and the subject is oriented in a semi-upright body position.

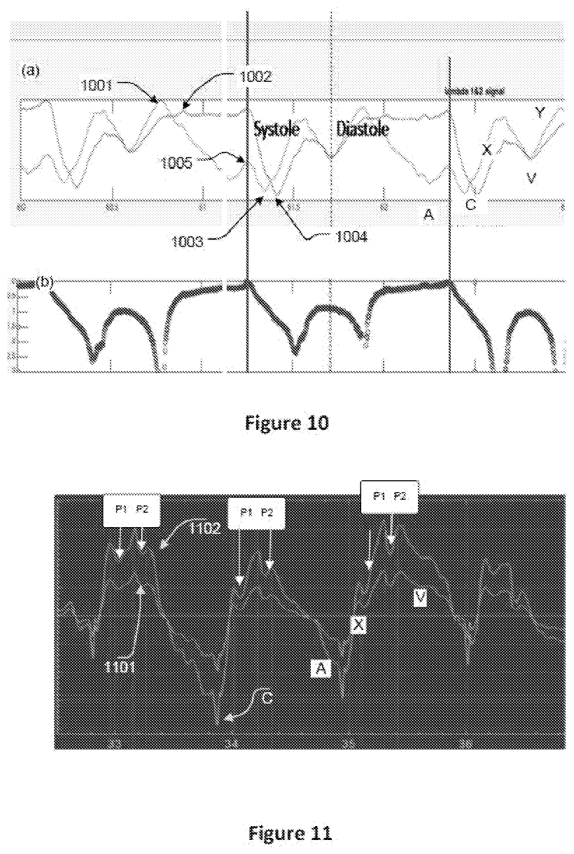

Referring to FIG. 10(a), an example plot of a first signal 1001 derived from measured light reflected from the internal organ at a first wavelength (e.g. 895 nm) and a second signal 1002 derived from measured light reflected from the internal organ at a second relatively shorter wavelength (e.g. 660 nm) is shown. These signals 1001, 1002 were obtained from an apparatus 100 placed on the human subject 520 in the vicinity of well-ventilated and perfused lung.

As shown in Figured 10(a), the first signal 1001 (895 nm) obtained from a ventilated lung is representative of a venous signal. The first signal 1001 comprises A-wave, C-wave, X-wave, V-wave, and Y-wave components.

Accordingly, in some embodiments, determining, by the processor, that at least one of the waveforms is representative of signals predominantly associated with the internal organ being a lung may comprise determining that at least one of the waveforms corresponds to a waveform of a venous pulse. As discussed above, determining that the at least one of the waveforms corresponds to a waveform of a venous pulse may comprise comparing the first and/or second waveforms to typical or measured venous pulse waveforms to determine a measure of conformity or comparing the first and/or second waveforms to measured arterial pulse waveforms and subject to determine a measure of non-conformity.

Again, and as discussed above, determining that at least one of the one or more signals is representative of signals predominantly associated with the internal organ may comprise comparing components of the waveform(s) of the one or more signals with template waveforms characteristic of the internal organ, in this case the lung, and may for example, involve de-convoluting the components from the waveform(s).

In some embodiments, the processor 562 may be configured to determine that a pulse shape of a waveform of a signal at a relatively long wavelength (e.g. about 895 nm) which is sensitive to oxygenated blood corresponds substantially with a characteristic lung pulse shape. For example, the characteristic lung pulse shape may comprise a waveform representative of a venous pulse signal and/or the characteristic lung pulse shape may comprise a first component with a generally increasing signal level (X-wave) at a first rate followed by a second component with a generally decreasing signal level (leading edge of the diastolic phase) at a second rate that has a smaller magnitude than the first rate.

In some embodiments, the processor 562 may be configured to determine that the waveform of the signal is representative of a signal from the lung when it is determined that the first waveform comprises an A-wave, X-wave, V-wave and Y-wave.

In some embodiments, the processor 562 may be configured to determine that a pulse shape of a waveform of a signal from light at a wavelength of about 660 nm, which is particularly sensitive to changes in deoxygenated blood levels, has a characteristic lung pulse shape. For example, the characteristic lung pulse shape may comprise a waveform representative of an inverted pulmonary artery pressure waveform (e.g. see Singal et al., J. Med. Devices 9(2), 020906, 2015, the disclosure of which is incorporated herein in its entirety by way of reference). The waveform may, for example, comprise a prominent systolic signal in the inverted pulmonary artery pressure waveform. The diastolic stage of the inverted pulmonary artery pressure waveform may also comprises a dicrotic notch that is similar in shape and timing to the V-wave in the waveform (marking the end of systole and the beginning of diastole). In addition, the start of the systolic pulse typically precedes the start of the skin arterial pulse. This may reflect the earlier contraction of the right ventricle in comparison to the left ventricle.

FIG. 21 shows two signals 2601 comprising a plurality of pulses obtained while the subject was breathing in region 2602 and then while the subject held their breath in region 2603. It can be seen that the waveforms (pulses) in region 2603 are more uniform in intensity ranges than the two signals 2601 in region 2602. In some embodiments, the subject may hold their breath while determining if the one or more signals are representative of signals from the lung. This may simplify and/or improve the accuracy of the determination.

### Signal Waveform—Liver

Referring to FIG. 11, an example plot of first and second signals 1101, 1102 received from an apparatus 100 placed on the human subject 520 in the vicinity of the liver is shown. The first signal 1101 signal is derived from measured light reflected from the liver at a first wavelength (e.g. 895 nm) and the second signal 1102 is derived from measured light reflected from the liver at a second relatively shorter wavelength (e.g. 660 nm).

As illustrated, A-wave, C-wave, and X-wave components may be observed in the first and second waveforms of the first and second signals 1101, 1102, respectively. In some embodiments, a V-wave component may also be observed. The waveforms of the first and second signals 1101, 1102 may also comprise additional small troughs (P-waves comprising P1-wave and P2-waves). The P1-wave may be indicative of a contribution to the first and second signals 1101, 1102 derived light reflected from the portal vein systolic pulse. The P2-wave may be indicative of a contribution to the first and second signals 1101, 1102 derived light reflected from the portal vein diastolic pulse. The presence of the P1 and/or P2 waves appear to be unique to signals from the liver; it is not observed in other organs.

Accordingly, in some embodiments, determining, by the processor 562, that at least one waveform of one or more signals derived from light reflected from the liver is representative of a signal predominantly associated with the liver may comprise determining that that the at least one waveform corresponds to a waveform of a venous pulse.

As discussed above, determining that at least one of the one or more signals is representative of signals predominantly associated with the internal organ being liver may comprise comparing components of the waveforms of the first and/or second signals with template waveforms characteristic of the liver, and may for example, involve de-convoluting the components from the waveform(s).

In some embodiments, determining, by the processor 562, that at least one of the waveforms is representative of signals predominantly associated with the internal organ being liver may comprise determining that at least one of the waveforms comprises at least one of an X-wave and a P-wave.

In some embodiments, the processor 562 may be configured to determine that a pulse shape of at least one of the one or more waveforms corresponds substantially with a characteristic liver pulse shape. For example, the characteristic liver pulse shape may comprise a waveform representative of a venous pulse signal and/or the characteristic liver pulse shape may comprise a first component with a generally increasing signal level (X-wave component) at a first rate followed by a second component with a generally decreasing signal level (leading edge of the diastolic phase) at a second rate that has a smaller magnitude than the first rate.

In some embodiments, the subject may hold their breath while determining if the one or more signals are representative of signals from the liver. This may simplify and/or improve the accuracy of the determination.

The P-wave component(s) for the liver trace may also lead to characteristic components in the calculated modified ratio of ratios and blood oxygen levels as discussed later. The processor 562 may determine that the signals are representative of signals from the liver based on the presence of these characteristic components.

### Signal Waveform—Intestine

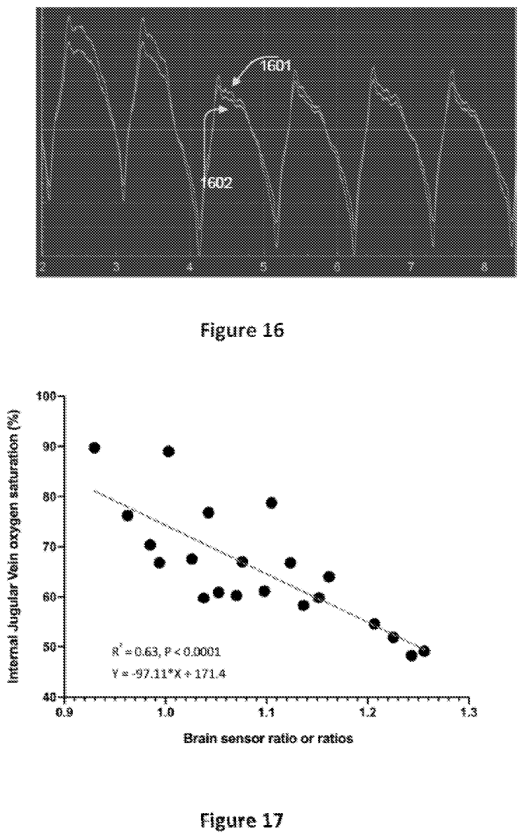

FIG. 16 shows an example plot of a first signal 1601 derived from measured light reflected from the intestines at a first wavelength (e.g. 895 nm) and a second signal 1602 derived from measured light reflected from the intestines at a second relatively shorter wavelength (e.g. 660 nm). The first and second signals 1601, 1602 were obtained from an apparatus 100 placed on the subject 520 in the vicinity of intestines.

The plethysmography signal derived from light reflected by intestines may be similar to the pressure changes in the central venous circulation with A, C, V, X and Y waves. These waves are temporally delayed relative to the skin and liver plethysmography signals. This may be due to the venous pulsation that must pass through the liver to reach the portal vein and then the microcirculation of the intestine. The delayed X-wave may therefore have a late convex component due to the simultaneous arrival of the arterial pulse of intestinal blood. The V-wave may not be prominent. Consequently the peak or maximum light intensity level of the pulse is very delayed and occurs in late systole relative to the forehead skin pulse.

Accordingly, the signal waveform for a signal from the intestine may demonstrate venous features with any one or more of A, C, X, V, Y waves and these waves may be delayed relative to the corresponding waves in the skin and liver. In some embodiments, determining, by the processor 562, that at least one of the waveforms is representative of signals predominantly associated with the intestine may comprise determining that the peak signal time of the one or more signals is offset from a peak signal time from a further signal such as a skin arterial signal. This offset (or lag) reflects the time for these pressure pulsations in the venous circulation to pass backward through the liver along the portal vein to reach the microcirculation of the intestine.

As with brain and liver, the quality of signals associated with the intestine may be improved if the subject holds their breath.

In some embodiments, the processor 562 may determine the health condition of an intestine based on a comparison with a characteristic waveform that is similar to what is illustrated in FIG. 16. A departure from the characteristic waveform may, for example, be used for diagnosis of any one or more of: ischaemic hepatitis, cirrhosis, abdominal compartment syndrome, portal vein thrombosis, portal vein hypertension.

### Signal Waveform—Kidney

Measured light reflected from the kidney at a first wavelength of about 895 nm and a second relatively shorter wavelength of about 660 nm may be used to determine kidney health. The first and second signals may be obtained from an apparatus 100 placed on the subject 520 in the vicinity of kidney.

The kidney has a very high arterial blood flow in comparison to other organs. The pulse shape of the waveform of the first signal from light at a wavelength of about 895 nm, is therefore not unexpectedly arterial in character with a prominent systolic minimum level. The waveform of the second signal from light at a wavelength of about 660 nm is relatively flat in comparison. However A, C, X, V and Y wave components are discernible. The flat waveform may be due to high oxygen levels throughout the pulse.

Accordingly, in some embodiments, determining, by the processor 562, that at least one a waveform of one or more received signals is representative of signals predominantly associated with the kidney comprises determining that the waveform is substantially arterial in character with a prominent systolic minimum level. In some embodiments, determining, by the processor 562, that at least one waveform of one or more received signals is representative of signals predominantly associated with the kidney comprises determining that the waveform is relatively flat but yet comprises A, C, X, V and Y wave components. In some embodiments, determining, by the processor 562, that at least one waveform of one or more received signals is representative of signals predominantly associated with the kidney comprises determining that the pulse shape of the waveform corresponds substantially with a characteristic kidney pulse shape.

### Signal Waveform—Foetal

The pulsatile signal arising from the foetus is distinct from the mother in a number of ways. These differences allow a straightforward method to confirm the signal is arising from the foetus. The heart rate is normally higher than the mother's heart rate, around 120-160 beats per minute compared to 70. The oxygen saturation levels are lower in the foetus. The arterial saturation in the foetal brain is low about 90% (mother 100%), other organs are even lower at around 65% (mother 100%). Consequently, venous blood oxygen saturation levels are very low between 25 and 40% (mother 75%). Finally, the shape of the pulse waveform is different as foetal blood pressure is very low and the circulation is functionally different to the mother's. The foetal brain provides an ideal target due to the high blood flow relative to the blood flow in the overlying tissues of the mother including the skin, abdominal muscle or cervix.

As the foetal pulse is not synchronous with its mother (biological or surrogate), the processor 562 may determine that the one or more signals are representative of signals from the foetus based on a comparison of peak signal levels of the one or more signals derived from light from the foetus with a peak signal level from a further signal derived from light from the mother.

### Signal Waveform—Muscle

Measured light reflected from the gastrocnemius muscle at rest from a human subject at a first wavelength of about 895 nm and at a second relatively shorter wavelength of about 660 nm can be used to assess muscle health. A third signal derived from light at a wavelength of about 895 nm and a fourth signal derived from light at a wavelength of about 660 nm from a simultaneous recording from the forehead skin from the subject may be utilised. At rest muscle blood flow is low and is therefore characterised by low pulse amplitude of the signal. The pulse shape is arterial in nature as skeletal muscle has relatively few venules relative to other organs.

Accordingly, in some embodiments, determining, by the processor 562, that at least one a waveform of one or more received signals is representative of signals predominantly associated with muscle comprises determining that the waveform determining that the pulse shape of the waveform corresponds substantially with a characteristic muscle pulse shape. In some embodiments, determining, by the processor 562, that at least one a waveform of one or more received signals is representative of signals predominantly associated with muscle comprises determining that the waveform depicts a signal with a relatively low pulse amplitude and which is substantially arterial in nature.

### Assess the Health of the Subject Based on the Waveform(s)

As discussed above with reference to FIG. 9, once the processor 562 determines that at least one of the one or more signals is predominately associated with light reflected from the targeted internal organ, one or more waveforms of the one or more signals may be analysed to determine or assess a health of the subject 520. For example, in some embodiments, at least one of the one or more waveforms may be compared with information characteristic of a health condition to assess the health of the subject 520.

For example, in some embodiments, one or more waveforms of the one or more received signals predominately associated with light reflected from the targeted internal organ may be compared with one or more characteristic pulse shapes of a respective healthy internal organ (for example, as may be stored as templates in memory). The processor may be configured to determine a measure of similarity (or dissimilarity) between the waveform(s) and characteristic pulse shapes and to determine or assess the health of the internal organ based on the measure of similarity (or dissimilarity).

Further described embodiments relate to assessing one or more waveforms to determine a likelihood of the subject having various conditions including disorders of organs associated with low microvascular blood oxygen levels or abnormal blood flow or abnormal movement. Examples include increased intra-cranial pressure (ICP), brain haemorhage, stroke, ischaemic hepatitis, pneumonia, ischaemic intestine, heart failure.

Pulsatile blood flow in organ's microcirculation may reflect the difference over the period of the cardiac cycle in the microvascular arteriolar pressure, which promotes blood flow, and microvascular venous pressure levels, which resists blood flow. The difference in microvascular pressure levels changes over the systolic and diastolic phases of the cardiac cycle and may define the shape and amplitude of the waveforms of signals derived from light reflected by the internal organs. A relative increase in systemic venous circulation pressure levels is associated with the plethysmography signal demonstrating predominately or exaggerated features similar to the pressure waveform of central veins. These features include A, C, X, V and Y waves with a peak signal level during the diastolic phase of the cardiac cycle. This finding may indicate low blood flow in the organ.

It may be useful to analyse the waveform derived from signals associated with light at a wavelength around 805 nm as it is not significantly influenced by changes in blood oxygen levels and is only influenced by blood flow. The waveform shape arising from a signal associated with a wavelength of about 805 nm may be used to detect abnormal patterns of blood flow due to disorders of the arterial and venous circulations. Such disorders of the systemic circulation that may be detected include heart failure and fluid overload which increases pressure levels in the venous circulation. Furthermore, central venous pressure levels may also be estimated non-invasively. For example, similar features may be seen in the waveform if the arterial blood pressure levels are low and the venous pressure levels normal. Disorders with this situation include vasospasm of cerebral arteries, thrombosis of arteries, as may occur in a stroke.

### Heart Failure

Measured light reflected from the brain of a human subject at a first wavelength of about 895 nm and a second wavelength of about 660 nm may be utilised to determine whether the human subject has heart failure. In this instance the first and second signals depict a prominent and early V-wave component.

To determine a likelihood of a subject having a heart failure, such as a leaky heart valve leading to tricuspid regurgitation, the processor 562 may analyse one or more waveforms of respective signals derived from detected light reflected from a brain 521. The processor 562 may derive data from the waveform(s) indicative of a V-wave component that is prominent and compare the derived data to a template waveform characteristic of a signal representative of a subject 520 suffering from heart failure to determine whether the derived data corresponds to the template waveform. Responsive to determining that it does correspond substantially (for example by a threshold amount) to the template waveform, the processor 562 determines that the subject 520 is likely to have heart failure This approach is applicable to a range of disorders that cause heart failure in which case the venous characteristics of the waveform will be prominent.

In some embodiments, the processor 562 may derive data from the waveform(s) indicative of the amplitude of the V-wave component. The processor 562 may compare the derived amplitude to a threshold level to determine if the subject is likely to have heart failure.

### Intra-Cranial Pressure (ICP)

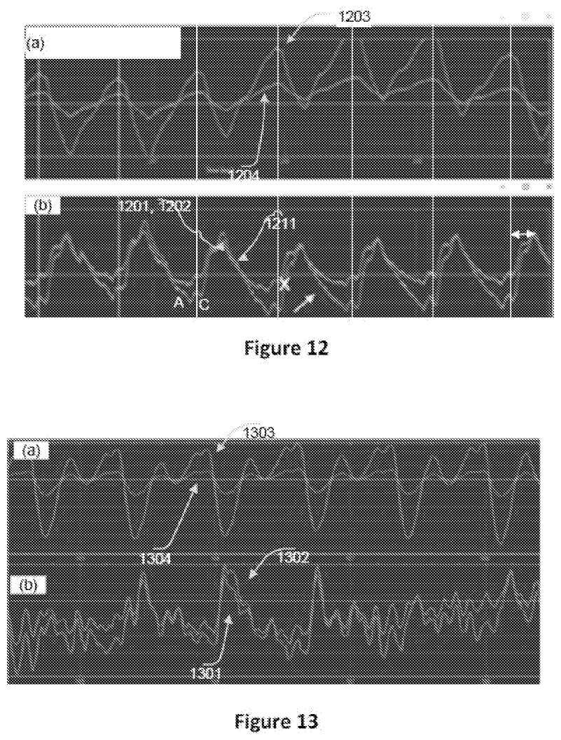

Referring to FIG. 12(b), an example plot of a first signal 1201 derived from measured light reflected from the brain of a subject (in this case, a sheep) at a first wavelength (e.g. 895 nm) and a second signal 1202 derived from measured light at a second relatively shorter wavelength (e.g. 660 nm) is shown. The first and second signals 1201, 1202 were obtained from sensors of the apparatus 100 placed on the scalp of an animal subject following an injection of 6 ml of blood into the anterior cranial fossa through a frontal burr hole to increase intra-cranial pressure in the sheep in the range of about 50 mmHg to around 90 mmHg. FIG. 12(a) shows an example plot of a third signal 1203 derived from light at a wavelength of about 895 nm and a fourth signal 1204 derived from light at a wavelength of about 660 nm obtained from the skin of the nose of the sheep, substantially simultaneously with the first and second signals of FIG. 12(a) and are representative of arterial skin signals.

For a healthy subject, it would be expected that the waveforms of the one or more signals would substantially correspond with a typical venous waveform. However, it can be seen that the first and second signals 1201, 1202 have waveforms that have components that appear more characteristic of an arterial waveform than a venous waveform. Specifically, the initial magnitude of the pulse slope (or gradient) of the leading edge (falling light intensity) 1211 of the pulse is greater than the initial magnitude of the slope of the leading edge 811 of a characteristic signal representative of a venous pulse 805, 806. In some cases, other venous features such as the V-wave and/or Y-wave component may be absent or diminished from the first and/or second waveforms or third waveforms (805 nm). It is clear this still represents a brain signal as there is a clear lag in the pulse onset compared to the skin signal, represented by the double ended arrow in FIG. 12 (b).

Thus, it has been determined that such a change in waveform to have some arterial features in shape may indicate an increase in intracranial pressure. This results from an increase in brain arterial pressure to maintain adequate blood flow, the pulse waveform develops a more arterial shape, as the arterial pressure is very much greater than the central venous pressure. These two pressures influence the shape of the pulse waveform.

Accordingly, in some embodiments, the processor 562 is configured to compare one or more waveforms of respective one or more signals derived from detected light from a brain 521 to a template waveform representative of a subject 520 experiencing relatively high intracranial pressure to determine if one or more of the waveforms is substantially representative of the template waveform. This may comprise, for example, calculating a sum of squares residual. The processor 562 may, responsive to determining that at least one of the waveform(s) is representative of the template waveform, determine that the subject 520 is likely to have relatively high intracranial pressure (e.g. if the intra-cranial pressure is less than about 90 mmHg). This may, for example, be indicative of development of brain odema or brain haemorrhage.

In some embodiments, the processor 562 may be configured to analyse at least one of a first component and a second component of the waveform(s) of respective signal(s) derived from detected light reflected from the brain 521 of a subject to determine a gradient of the leading edges (see for example 1211 of FIG. 12) of the respective of the initial slope of the pulse. Responsive to determining that at least one of the gradients of the leading edges is greater than a threshold value, the processor 562 may determine that the subject 520 is likely to have relatively high intracranial pressure.

In some embodiments, the processor 562 is configured to analyse at least one of a first component and a second component of the waveform(s) of respective signal(s) derived from detected light from a brain 521 to determine if a V-wave and/or Y-wave component is present. Responsive to determining that a V-wave and/or Y-wave component is absent, the processor 562 may determine that the subject 520 is likely to have relatively high intracranial pressure.

Disorders Associated with Relatively Raised Pulmonary or Systemic Arterial Circulation Pressure Levels

Disorders associated with relative increase in pulmonary arterial circulation pressure levels include pulmonary embolus, interstitial lung disease, chronic obstructive lung disease, pneumonitis, acute lung injury and/or acute respiratory distress syndrome. A relative increase in pulmonary arterial pressure levels compared with pulmonary venous pressure levels is associated with a lung plethysmography pulse signal 1001 (e.g. from light with wavelengths of 895 nm or 805 nm) demonstrating some features of the pulmonary arterial circulation pressure waveform, such as the A, C, V and Y wave components, becoming less prominent and components that are similar to features from a pulmonary artery pressure waveform developing, including a prominent systolic pulse, a diastolic pulse and also a dicrotic notch (which may coincide with a V-wave and Y-wave).

In some embodiments, the processor 562 may analyse the shape of the waveform(s) of the signal(s) derived from detected light from the target internal organ to determine health conditions associated with a relative increase in systemic arterial circulation pressure levels compared to central venous pressure levels, such as systemic hypertension and regional organ disorders such as acute brain injury, liver cirrhosis and/or compartment syndromes. For example, the processor may be configured to compare the waveforms(s) or data from the waveform(s) with template waveforms or data characteristic of such health conditions to determine a likelihood of the subject exhibiting the health condition.

Disorders associated with raised arterial pressure levels may also be detected such as hypertension in which case the waveform develops a more arterial pulse shape. Other disorders include raised intra-cranial pressure in the brain and compartment syndromes elsewhere in the body, such as abdominal or calf compartment syndromes. The same approach may be used for the pulse or waveform arising from the microcirculation of the lung to detect abnormal waveforms that may due to disorders resulting in raised pulmonary arterial pressure levels (interstitial lung injury, ARDS, pulmonary embolus) or raised pulmonary vein pressure levels (left sided heart failure).

Disorders Associated with Relatively Raised Systemic Venous Circulation Pressure Levels

A relative increase in systemic venous circulation pressure levels may be associated with disorders including heart failure and fluid overload secondary to intra-venous fluid administration. Monitoring the waveform shape may assist in monitoring systemic venous circulation pressure levels to detect heart failure and guide intra-venous fluid resuscitation of the circulation to avoid fluid overload.

A relative increase in systemic venous circulation pressure levels is associated with a waveform of 660 nm or 805 nm demonstrating more prominent or exaggerated features of the pressure waveform of central veins. These features include A, C, and V wave components with a minimum signal value, and X and Y wave components with an increasing signal value.

The processor 562 may, for example, determine that the subject has such a disorder from a determination that the magnitude or amplitude of any one or more of the A-wave, V-wave, X-wave and/or C-wave components is greater than a threshold value. The magnitude or amplitude of the component may be calculated as the range between the maximum and minimum values of the component.

### High Intracranial Pressure

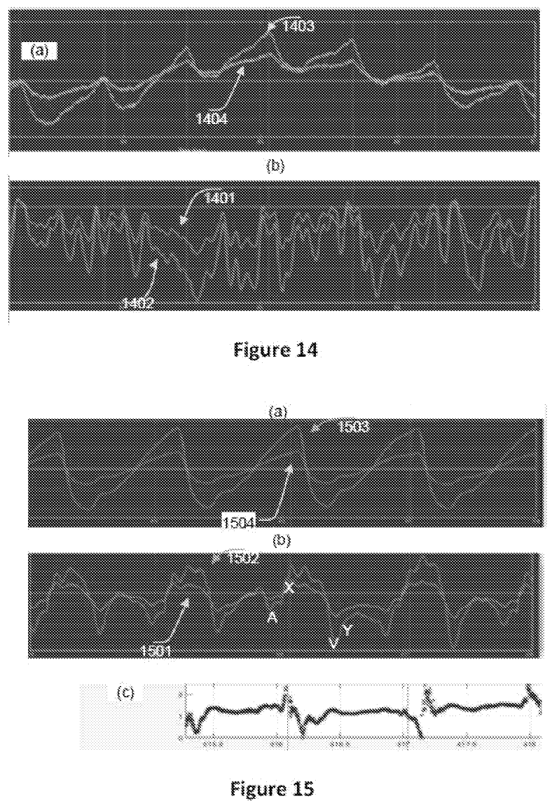

Referring to FIG. 14(b), an example plot of a first signal 1401 derived from measured light reflected from a brain of a subject at a first wavelength (e.g. 895 nm) and a second signal 1402 derived from measured light reflected from the brain at a second relatively shorter wavelength (e.g. 660 nm) is shown. The first and second signals 1401, 1402 were obtained from an apparatus 100 placed on the scalp of a sheep following an injection of 6 ml of blood into the anterior cranial fossa through a frontal burr hole to increase intra-cranial pressure in the sheep in the range of greater than about 150 mmHg. FIG. 14(a) shows the simultaneous third and fourth signals 1403, 1404 obtained from the nose skin of the subject 520 and are shown for comparison.

As illustrated, the first and second signals 1401, 1402 show an increase in the amplitude of the AC pulse signal, which is characteristic of intra-cranial pressure in the subject.

If the pulse amplitude increases in the brain signal, but does not change in the skin signal, this indicates that the disorder is likely to be confined to the brain. An example is an isolated increase in blood flow to the brain in response to an increase in intra-cranial pressure (ICP) levels. In which case, the amplitude of the pulse (signal level) increases on the brain signal, but as skin blood flow has not changed, there is no such change in the skin pulse amplitude. On the other hand, if the pulse amplitude increases at both sites (skin and brain) this indicates the disorder is likely to be effecting all parts of the body. An example is low oxygen levels throughout the body, with an increase in blood flow throughout the body to compensate, this will cause changes in the pulse waveform throughout the body. This approach applies to detecting disorders in other organs of the body.

The processor 562 may analyse at least one waveform of respective one or more signals derived from detected light from a brain 521 to determine if the waveforms show that an AC component amplitude has increased relative to a corresponding component of an arterial waveform of a further signal obtained simultaneously with the one or more signals from the subject, such as a skin signal. Responsive to determining that the AC component amplitude has increased, the processor 562 may determine that the subject 520 is likely to have a relatively high intracranial pressure or brain haemorrhage. In addition, a drop in the DC level or brain microvascular blood oxygen levels followed by an increase may also indicate the subject is suffering from high intra-cranial pressure levels or a brain haemorrhage.

### Poorly Ventilated Lung

In poorly ventilated lungs pulmonary arterial blood flow is low. Pulmonary vein pressure levels are therefore relatively high compared to the pulmonary artery pressures experienced by this region of the lung and the waveform of the sensor reflects this, with a predominately venous signal (FIG. 15 (b)).

To obtain one or more signals predominantly associated with the lung, the apparatus 100 may be located on the back of the subject 520 while the subject is lying supine with atelectatic (collapsed) alveolar air sacs.

Referring to FIG. 15(b), an example plot of a first signal 1501 derived from light at a longer wavelength (e.g. 895 nm) and a second signal 1502 derived from light at a shorter wavelength (e.g. 660 nm) are shown. The first and second signals 1501, 1502 were obtained from an apparatus 100 placed on the subject 520 in the vicinity of dependent poorly ventilated lung. FIG. 15(a) shows the simultaneous third and fourth signals 1503, 1504 obtained from the forehead skin of the subject 520 which are shown for comparison.

As illustrated the first signal 1501 and the second signal 1502 may comprise components consistent with the pressure waveform found in pulmonary veins. These features include A, C, X, V and Y waves. The V-wave is prominent in the waveform of the second signal 1502 but less prominent in the waveform of the first signal 1501. The minimum signal value for the first signal 1501 and the second signal 1502 may occur during the V-wave rather than the A-wave. The peak signal value is synchronous for the first signal 1501 and the second signal 1502. The processor 562 may determine that the health condition comprises a poorly ventilated lung based on any one or more of these features.

For example, in some embodiments, the processor 562 may be configured to compare data derived from the second waveform, i.e., the signal associated with a wavelength at about 660 nm to a characteristic waveform of a poorly ventilated lung, which depicts a prominent V-wave component. Responsive to determining that the waveform is representative of the characteristic waveform with a prominent V-wave, the processor may determine that the health condition comprises a poorly ventilated lung.

It is noted that oxygen levels remain relatively low and constant during systole and diastole as oxygen is not added to a significant extent to the blood as the air sacs are collapsed (see FIG. 15(c)). Determining oxygen levels is discussed in further detail below.

### Hypoxia—Liver

Changes in portal vein blood flow are important in detection of a range of liver disorders including hepatitis, cirrhosis and right heart failure.

Under normal oxygenation, the P-wave may be insignificant or not observable in the signals. However, with the development of systemic hypoxia, the P wave becomes very prominent due to increased cardiac output with increased portal vein blood flow. The dominance of the P-wave leads to the X-wave not being observed. Note also, that the pulse rate is higher under hypoxic conditions.

Accordingly, in some embodiments, responsive to determining that at least one waveform of one or more signals derived from light reflected by the liver differs from a template waveform representative of a healthy liver, the processor 562 may determine that the subject suffers from any one or more of hepatitis, cirrhosis and right heart failure. In some embodiments, responsive to determining that at least one waveform of one or more signals derived from light reflected by the liver exhibits a dominant P-wave component, and optionally no X-wave component, and optionally, an increased pulse rate, the processor 562 may determine that the subject has increased portal vein blood flow.

### Intra-Cranial Pressure—Sylvian Fissure

A first signal can be derived from detected light at a wavelength of about 660 nm that is reflected from movement of the cerebral spinal fluid located in the Sylvian fissure of a human subject. In this context a third signal derived from light at a wavelength of about 660 nm obtained from the forehead skin of the subject is representative of an arterial pulse from the subject. The first waveform of the first signal has a similar shape to the waveform of the third signal, that is an arterial waveform. The first waveform of the first signal may also comprise particular oscillations which are indicative of a signal arising from the movement of the cerebral spinal fluid located in the Sylvian fissure.

The timing of the particular oscillations and the general shape of the first waveform of the first signal correlates closely with the observed waveforms documented for intra-cranial pressure measurements in the cerebral spinal fluid that result from pressure changes in the skull following each arterial pulse of blood to the brain. (see Zweifel, C., Hutchinson, P., & Czosnyka, M., 2011; Intracranial pressure; in B. Matta, D. Menon, & M. Smith (Eds.), Core Topics in Neuroanaesthesia and Neurointensive Care, pp. 45-62, the disclosures of which are incorporated herein in their entirety by way of reference). Of note, the peak intensity level (which can be used as the start of the waveform or pulse) occurs slightly before the forehead skin pulse, which is consistent with the pulse representing the effect of pressure changes in the brain on cerebral spinal fluid movement rather from blood flow in the microcirculation of the brain. The pattern, amplitude and frequency of the oscillations may be used to detect abnormally raised intracranial pressure levels.

In some embodiments, the apparatus 100 is located adjacent to a sulcus to enable non-invasive monitoring to detect raised intra-cranial pressure levels. Light comprising at least one wavelength from the light source 120 may be projected through the cranium of the subject 520 to the sulcus. The light reflected from cerebrospinal fluid (CSF) in the sulcus may be received at a photodetector 130 of the apparatus 100. The processor 562 may then produce at least one signal derived from the measured light reflected from the sulcus at the respective wavelength. The waveform(s) of the at least one signal may be analysed to determine a measure of raised intra-cranial pressure. For example, one or more of the signals may comprise a superimposed oscillatory signal. The frequency and amplitude of the oscillatory signal may increase with raised intracranial pressure. Responsive to determining that the frequency or amplitude of the oscillatory signal is above a threshold level, the processor 562 may be configured to determine that the subject is experiencing raised intracranial pressure.

If blood is present in the CSF, as occurs with sub-arachnoid haemorrhage, the signal levels of the signals may become very strong compared to signals from normal CSF. The signals may therefore be used to detect sub-arachnoid haemorrhage.

In some embodiments, the apparatus 100 produces and detects light at a wavelength of about 805 nm to produce the first signal and reduce the effect of blood oxygen levels on the first signal. This may lead to a more accurate and/or reliable waveform shape that reflects the ICP.

### Organ Movement—Brain

Referring to FIG. 13(b), an example plot of a first signal 1301 derived from measured light reflected from a brain of a subject at a first wavelength (e.g. 895 nm) and a second signal 1302 derived from measured light at a second relatively shorter wavelength (e.g. 660 nm) are shown. The first and second signals 1301, 1302 were obtained from sensors of the apparatus 100 placed on the scalp of a sheep following an injection of 6 ml of blood into the anterior cranial fossa through a frontal burr hole to increase intra-cranial pressure in the sheep in the range of about 90 mmHg to about 150 mmHg. It can be seen that the first and second waveforms associated with signals 1201, 1202 comprise a high amplitude oscillation at a particular frequency, in this case of about 7 Hz. Oscillations, at this frequency, in ICP pressure tracings have previously been documented and represent “ringing” of the brain in response to the systolic arterial pressure wave entering the brain. Increased ICP pressure levels may increase the amplitude of these oscillations giving rise to the changes in the monitor's waveform we detected. The synchronous nature of the monitors pulsations with the high frequency oscillations are consistent with both triggered by a cardiac source. Previous clinical studies, in the setting of brain injury, found high frequency and amplitude oscillations in the ICP were associated with poorer patient outcomes.

FIG. 13(a) shows a simultaneous plot of third and fourth signals 1303, 1304 obtained from the nose skin of the sheep and depicting an arterial waveform shown for comparison to the first and second signals 1301, 1302. This may assist in determining the timing of the systolic and diastolic phases of the cardiac cycle.

The processor 562 may analyse at least one waveform of respective one or more signals derived from detected light reflected from a brain 521 to determine if the waveform comprises an oscillation, for example, at about 7 Hz. Responsive to determining that the waveform(s) comprise the oscillation, the processor 562 may determine that the subject 520 is likely to have a relatively very high intracranial pressure. The very high intracranial pressure may, for example, be caused by an acute brain injury due to a brain haemorrhage.

### Organ Movement—Lung

The lungs expand during inspiration and contract during expiration. The optical signal from the lungs reflects the extent of this movement with breathing. The optical signal can be used to detect abnormal patterns of breathing and the phases of breathing. The optical signal detects the inspiratory and expiratory phases of respiration. This signal may be used to trigger mechanical ventilators. Accordingly, the processor 562 may be configured to output a control instruction to control a mechanical ventilator based on a determined breathing pattern.

Organ movement—liver The liver sits under the diaphragm of the lungs and moves during breathing. First and second signals can be derived from light reflected by the liver of a subject 520 using apparatus 100 while the subject 520 is breathing over several respiratory cycles. The signal levels of the first and second signals change with the inspiratory and expiratory phases of respiration and accordingly, the first and/or second signals derived from the liver can be used to detect abnormal patterns of breathing and the phases of breathing. The lower frequency respiratory oscillation is much greater in amplitude compared to the higher frequency cardiac oscillations. Inspiration is associated with a rapid drop in the signal levels (transmitted light intensity) of the first and second signals and expiration is associated with an increase in the signal levels.

In some embodiments, detection of abnormal patterns and/or phases of breathing may be used to trigger a mechanical ventilator coupled to the subject and/or to detect disorders of the liver such as cirrhosis.

For example, the processor 562 may be configured to compare changes in the inspiratory and/or expiratory phases of respiration depicted in a waveform of a signal derived from light reflected by the liver of a subject 520 with corresponding changes in the inspiratory and/or expiratory phases of respiration depicted in a template waveform characteristic of a relatively healthy liver. The waveform(s) and template waveform may extend over at least one respiratory cycle. The respiratory cycle may span a time range of over 1 second and may for example, span a time range from about 1 second to about 10 seconds.

In some embodiments, the processor 562 may be configured to determine movement of the liver based on a statistical measure of the at least one waveform. The statistical measure can be compared to the information characteristic to determine the movement. The statistical measure may comprise any one or more of: a peak signal value, a minimum signal value, a median signal value, a root-mean square signal value, and an average signal value of a waveform of the first and the second signal. The processor 562 may, for example, determine a range of signal levels from the difference between the peak signal value and minimum signal value may. The processor 562 may determine that the range of signal levels is representative of either a healthy or unhealthy condition. For example, the processor 562 may determine that the range of signal levels is outside a threshold range or beyond a threshold value.

### Organ Movement—Heart

The movement of the heart may be used to detect the phases of right and left ventricle systole and diastole and also atrial contraction, which may be used to detect abnormalities of heart function including systolic and diastolic heart failure also electrical conduction disorders.

A first signal derived from measured light reflected from a heart of a healthy human subject at a first wavelength (e.g. 895 nm) and a second signal derived from measured light at a second relatively shorter wavelength (e.g. 660 nm) may be utilised to detect heart movement. The apparatus 100 is placed on the anterior chest adjacent to the right ventricle of the heart. The right ventricle of the heart is a midline structure that is in direct contact with the chest wall lying below the sternum. A third signal may be derived from light at a wavelength of about 895 nm and a fourth signal derived from light at a wavelength of about 660 nm from the forehead skin, and a fifth signal from the right internal jugular vein of the subject.

In some embodiments, the processor may be configured to analyse a first waveform and second waveform of the first signal 3101 and the second signal 3102, respectively, to determine abnormal movements of the heart and/or the timing in the cardiac cycle of the heart chambers contractions and relaxations. The heart has 4 chambers right and left atria and right and left ventricles. They contract in that sequence. The waveforms of the first and second signals demonstrate the timing of each chamber's contraction in the cardiac cycle and also the relaxations for the right and left ventricle. Accordingly, abnormal movements of the heart as may occur following an injury to the heart due to damage to the muscle from a myocardial infarct or other cause of heart failure such as chronic hypertension may be detected by analysis of the waveforms. The waveforms may also be analysed to detect abnormal timing in the contraction of the cardiac chambers, as may occur with disorders of the conduction of the electrical signals that coordinate activation of cardiac muscle contraction.

### Analysis of Blood Oxygen Levels Using Modified Ratio of Ratios Calculation

In some embodiments, the processor 562 is configured to determine blood oxygen levels of the subject based on an analysis of first and second waveforms of respective first and second signals derived from light reflected by a targeted internal organ of a subject 520, which may be indicative of the health of the subject. The intensity of light reflected by blood within blood vessels is affected by blood oxygen levels. The absorption may be based on the detected intensity of first and second signals at respective first and second wavelengths using known methods of calibration for the intensity of the produced light. The blood oxygen levels correlate with a ratio of ratios R based on the intensities (or absorption) of the signals at two wavelengths.

For conventional pulse oximetry based on light signals from skin arterial blood, the ratio of ratios R is normalised based on a maximum signal value at the start of the pulse (relating to the maximum intensity of received light), also known as the DC level. The maximum intensity corresponds with the start of the systolic stage of the cardiac cycle.

Signals at each wavelength are normalised based on the maximum intensity at the respective wavelength. The conventional ratio of ratios R is given by:

$${R}^{′} = \frac{( \frac{A {C}_{1}}{{I}_{1}} )}{( \frac{A {C}_{2}}{{I}_{2}} )}$$

where I₁ is the signal value at start of the pulse (peak or maximum signal value) of signal 1 derived from received light at the shorter wavelength 1, AC₁ is the change in the signal level from I₁ at the peak of the systolic stage (minimum signal value which corresponds with peak or maximum of the blood pulse) of signal 1, derived from received light at the wavelength 1, I₂ is the signal value at start of the pulse (peak or maximum signal value) of signal 2 derived from received light at the longer wavelength 2, AC₂ is the change in the signal level from I₂ at the peak of the systolic stage (minimum signal value which corresponds with peak or maximum of the blood pulse) of signal 2, derived from received light at the wavelength 2. As a result, a single ratio of ratios value R′ is obtained for each waveform (or each pulse).

To provide useful results for assessing the health of an internal organ of a subject, the ratio of ratios is modified. The modified ratio of ratios equation accounts for some of the features of microvascular blood in internal organs that make it different to arterial blood in the skin.

When calculating a ratio of ratios values from signals derived from internal organs, the signal values may need to be normalised in a modified manner due to the unique features of microvascular blood in internal organs that make it quite different to known methods used in skin pulse oximetry used to calculate R′ and measure arterial blood oxygen levels.

In organs, microvascular blood oxygen levels change a great deal over the cardiac cycle, due to exchange of oxygen with the tissues of the organ. Levels are higher in microvascular blood during systole and fall during diastole (reverse for lungs); consequently the temporal point of the maximum transmitted light intensity values (start of pulse) may not be synchronous for both wavelengths. In skin pulse oximetry oxygen levels usually remain more constant and the temporal point of the maximum transmitted light intensity values (start of pulse) are always synchronous for both wavelengths.

In organs, the minimum light intensity level occurs later in the pulse (waveform) during diastole, particularly for signals derived from light at a wavelength of 660 nm (or 895 nm for lungs). This typically occurs during the A wave of the diastolic phase of the cardiac cycle. In contrast, in skin pulse oximetry the minimum light intensity level occurs during systole.

The minimum blood oxygen levels measured during diastolic phase of the cardiac cycle have particular importance, as the oxygen concentration in the blood of the microvasculature may have fallen to the point of being in equilibrium with the organ's extravascular tissue's oxygen concentration. This level may therefore provide an estimate of the oxygen level in the extravascular tissues of the organ. An exception is the lung in which the minimum oxygen levels occur during systole and represents the oxygen level in the mixed venous blood returning to the lung.

The peak oxygen levels measured during the systolic phase provide an estimate of the arterial oxygen levels in the microvasculature blood of organs. An exception is the lungs in which the peak oxygen levels occur during diastole and represent the extent to which the lungs have oxygenated the returning venous blood. It provides an estimate of the systemic arterial oxygen levels.

To address these differences and enable measurement of the diastolic phase oxygen levels, the modified ratio of ratios is calculated throughout the entire cardiac cycle to allow the change in oxygen levels over this entire period to be measured. The fall in oxygen levels during the diastolic phase of the cardiac cycle provides important clinical information. In contrast, with skin pulse oximetry, only the oxygen levels during systole are measured and reported.

Monitoring of diastolic oxygen levels is of particular importance as accurate non-invasive methods were not previously available. Diastolic oxygen levels reflect tissue oxygen levels and is of fundamental importance, as even short periods of low tissue oxygen can cause tissue necrosis and risks the development of organ failure and death. Monitoring of diastolic oxygen levels, therefore, allows early detection and treatment of systemic disorders including sepsis, heart failure and haemorrhage and may also be used to optimize treatment with fluid resuscitation and inotrope administration. In addition, monitoring of diastolic oxygen levels may provide early detection and treatment for organ specific disorders such as raised intra-cranial pressure, stroke, cerebral vasospasm, encephalitis, ischaemic hepatitis, ischaemic gut, nephritis, hepatitis, colitis and inflammatory bowel disease and abdominal and muscle compartment syndromes and pneumonitis.

To perform the modified ratio of ratios calculation (R), first and second waveforms associated with respective signals dominated by measured light reflected from the internal organ at respect distinct wavelengths are required. For example, the first wavelength may be at about 660 nm and the second wavelength may be about 895 nm. Determining modified ratio of ratios values for the waveforms comprises determining a plurality of signal level values across a window of the waveforms that corresponds to a cardiac cycle of the subject and normalising those values. The maximum signal value at each wavelength at the start of the pulse or waveform (the DC level) is of particular importance in the calculation of R. It is used to both normalise the signals and also assess the change in the light intensity level during the pulse (known as the AC level).

The modified ratio of ratios R values are indicative of the blood oxygen level values of the internal organ throughout the cardiac cycle or pulse of the subject. In some embodiments, the modified ratio of ratios R at a given time point t during the systolic and diastolic phases of waveform, may be calculated as follows:

$$R ( t ) = \frac{( \frac{A C 1 ( t )}{{I}_{1} ( {t}_{0} )} )}{( \frac{A C 2 ( t )}{{I}_{2} ( {t}_{0} )} )}$$

where AC₁(t) is the change in the signal level of the shorter wavelength signal from I₁ at time t, AC₂(t) is the change in the signal value of the longer wavelength signal from 12 at time t, and I₁(t₀) is the signal value at start of the pulse (peak or maximum signal value) of signal 1. I₁(t₀) is used as the first normalisation factor for the shorter wavelength, and this temporal point defines t₀. I₂(t₀) is the signal value of the longer wavelength signal at t₀ and this is used for the second normalisation factor for the longer wavelength signal. Determination of suitable time t₀ is described below.

Modified ratio of ratios R values are calculated for all values of t throughout the window of the waveform that corresponding with the cardiac cycle or pulse of the subject to thereby allow for monitoring of oxygen levels during both the systolic and diastolic phases of the pulse, unlike a conventional approach. In some embodiments, the processor 562 may determine the modified ratio of ratios R at a plurality of times throughout a window of the one or more waveforms corresponding with the cardiac cycle. The signal may, for example, be sampled at a sampling rate greater than the pulse rate (or heart rate) to provide a plurality of signal values throughout the waveforms. The signal may be sampled at a sample rate greater than 5 Hz. In some embodiments, the sampling rate may be in the range of 100 Hz to 5000 Hz. The sampling rate may be 500 Hz. The sampling rate may be sufficiently high so as to be considered substantially continuous. This provides a measure of the change in oxygen levels throughout all phases of the cardiac cycle.

As mentioned, in organs, the temporal locations for the peak signal value (maximum transmitted light intensity, the absolute signal levels or DC level) for the two or more signals may not be synchronous, with the absolute signal level for the second signal (at a shorter wavelength such as 660 nm) occurring later. In embodiments when the start of the waveforms (or pulses) of the two signals are not synchronous, the normalisation factors may be determined at time t₀, which corresponds with the peak light intensity signal value of the first or second signal.

The calculated modified ratio of ratios R may not be accurate if there is a variation in the signal value such that the intensity I(t) during the pulse is greater than or equal to the normalisation factors used. Unlike with skin, the presence of the low frequency respiratory oscillations in the plethysmography signal may prevent calculation of the ratio of ratios using the traditional approach, particularly for the period of the pulse during the diastolic phase. This can occur during the lower thoracic pressure phase of the respiratory cycle (due to venous blood draining more rapidly from the organ during this phase), and consequently the transmitted light intensity level during diastole may actually exceed the peak signal value (such as maximum light intensity) at the start of the pulse. This leads to a negative value for the AC level during diastole, making calculation of the modified ratio of ratios R not possible. This prevents use of a conventional approach in this situation.

To address the limitation of the approach described above for calculating the modified ratio of ratios R, when during the systolic or diastolic phases of the pulse the light intensity exceeds the level at the start of the pulse (I), (for example due to breathing), the maximum signal value of the next pulse 3208 is used in the calculations to derive the modified ratio of ratios R of the previous pulse (a backward normalisation approach). Accordingly, in some embodiments, the time t₀ where the normalisation factor is determined may represent the light intensity values at to of the following pulse to calculate the modified ratio of ratios of the previous pulse. We will define this time point as t₀₊₁ for this approach.

Another approach to deal with the issue of signal values during the pulse increasing above an earlier peak signal value is to undertake a forward normalisation approach (i.e. still use the peak signal value at the start of the pulse t₀), but provide the modified ratio of ratios R values calculated separately for each of the phases of the respiratory cycle (inspiration, inspiratory pause, expiration and the expiratory pause). These phases can be identified by the lower frequency oscillations in the plethysmography signal occurring at the frequency of the respiratory rate.

Another approach, particularly with regard to the liver and lungs, where the effect on the signal values during breathing the respiratory oscillation amplitude is very much greater than the cardiac oscillation, is to evaluate the calculated modified ratio of ratios R based on the respiratory oscillations in the signal rather than the cardiac oscillations, where t_{0R} represents the peak signal value at the start of the respiratory oscillation for each wavelength. We will define this time point as t_{0R} for this approach. We will call this a respiratory normalisation approach.

It has been found the temporal distance (or time offset) between the peak signals is proportional to the extent of difference in the systolic and diastolic oxygen levels and can be used to estimate the diastolic oxygen levels, as the arterial or systolic oxygen levels are usually known (through conventional methods). For example, in some embodiments, the processor may be configured to determine a temporal distance between the peak values of the first and second waveforms associated with signals predominantly associated with a targeted internal organ and to determine diastolic oxygen levels of the internal organ based on the temporal distance and associated arterial or systolic oxygen levels. In the case of the lungs, the diastolic or arterial oxygen levels are usually known and therefore the mixed venous oxygen levels may be estimated.

The minimum microvascular blood oxygen saturations that occur during diastole were found to also vary with the phase of respiratory cycle, with levels falling further during the expiratory phase. The lower levels reached during expiration reflect reduced oxygenation of the blood during this phase of ventilation by the lungs, as the air sacs may collapse during expiration. This oscillation in the minimum oxygen levels in the microvascular blood of organs associated with the respiratory phase may be used to assess the extent of oxygen exchange by the lungs with the blood circulation. This knowledge can be used to detect disorders of the lungs and to adjust mechanical ventilation parameters such as the positive end expiratory pressure level in patients to improve oxygen exchange in the lungs.

Referring again to the method 900 of assessing the health of a subject of FIG. 9, in some embodiments, the processor 562 processes the one or more received signals to remove a DC offset to produce AC signals for further analysis. The modified ratio of ratios may be determined from the AC signals. The signals may be an analogue electrical signal that is digitally sampled (digitised) using an analogue to digital converter (not shown).

In some embodiments, the processor 562 may be configured to determine that the first and second signals are associated with the internal organ based on a comparison of the determined modified ratio of ratios with a characteristic waveform or a template. The characteristic waveform or a template may, for example comprise a modified ratio of ratios determined from arterial skin signals. The modified ratio of ratios from arterial skin signals does not vary much from a value of around 0.6 for the majority of the pulse (or cardiac cycle phases of systole and diastole). The processor 562 may therefore determine that modified ratio of ratio values that differs from this by greater than a threshold amount, for example by 0.2, are indicative of the first and second signals (from which the modified ratio of ratios is calculated) being predominately associated with the internal organ. For example, if an average of the determined modified ratio of ratio values is greater than about 0.7, the processor may be configured to determine that the first and second signals are predominantly associated with the targeted internal organ. This is discussed with respect to specific examples of internal organs below. A template may also be used that represents the characteristic temporal changes in the modified ratio of ratio's over the systolic and diastolic phases for a given internal organ.

In some embodiments, the processor 562 may be configured to analyse the variation of the determined blood oxygen value to determine if the blood oxygen values are indicative of those of an internal organ. In some embodiments, responsive to determining that the blood oxygen levels are relatively high at the peak of at least one of the two waveforms, and fall as the two or more signal levels decrease, the processor 562 may determines that the blood oxygen levels are indicative of blood oxygen levels from the internal organ.

In some embodiments, the processor 562 may assess the health of the internal organ based on determined modified ratio of ratios calculations. For example, the processor 562 may determine blood oxygen levels of the subject using the modified ratio of ratios equation and may make health assessments based on the determine blood oxygen levels and the targeted internal organ.

For example, the processor 562 may determine the blood oxygen levels from the determined modified ratio of ratios values by comparing the modified ratio of ratios values to a look-up table or applying an empirically determined equation. The data for the look-up table or the data from which the empirical equation is determined may be based on concurrent measurements using conventional oximetry techniques.

In some embodiments, the processor 562 may determine blood oxygen levels throughout the two or more waveforms (e.g. for each respective modified ratio of ratios value determined or at least a subset of them). Moreover, the blood oxygen levels may be determined over the entire systolic and diastolic periods of each cardiac cycle (or pulse) to enable monitoring of the change in oxygen levels in the microvascular blood due to oxygen exchange with the tissues of the organ. The processor 562 may determine the blood oxygen level of the internal organ of the subject 520 based on the determined blood oxygen levels at a particular time in the waveform.

The minimum signal level of the first and second signals may coincide with when the blood oxygen levels in the microcirculation have reached equilibrium with the tissue levels in the internal organ. Accordingly, in some embodiments, the processor 562 may determine an equilibrium blood oxygen level value of the internal organ by determining a signal value of the first or second signal when the signal is at a minimum level. However, in some cases, blood oxygen levels may not reach equilibrium with the tissue oxygen levels. A non-equilibrium condition may occur if the signal arises predominately from a capillary bed, or if there is a very high blood flow, or shunting. The capillary bed signal is distinctive and different to that from venules.

FIG. 17 is an example plot of the oxygen saturation of the internal jugular vein of three human subjects during systemic hypoxia trials plotted against the modified ratio of ratios determined from applying the apparatus 100 to obtain signals from the brain of the three human subjects. The blood was aspirated from the vein placed in a blood gas machine to determine the oxygen saturation of the blood. The blood oxygen level in the vein was shown to be inversely proportional to the modified ratio of ratios. This demonstrates that the modified ratio of ratios calculated from signals derived from light reflected by the human brain can be easily mapped to oxygen levels in venous blood draining from the brain. It also provides evidence that the modified ratio of ratios values may be used to determine blood oxygen levels associated with an internal organ.

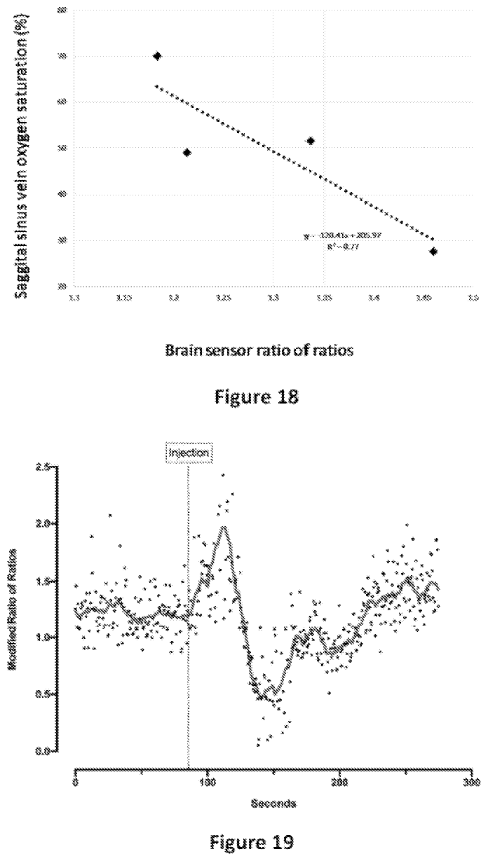

FIG. 18 is a plot of the oxygen saturation of the sagittal sinus vein of a single sheep during a brain injury trial in which blood was injected directly into the skull to change intracranial pressure and reduce blood flow and tissue oxygen levels. The sagittal sinus vein drains blood from the vein. The plot of FIG. 18 demonstrates the modified ratio of ratios determined from applying the apparatus 100 to obtain signals from the brain of the sheep. The blood oxygen level is inversely proportional to the modified ratio of ratios. This provides further evidence that modified ratio of ratios values can be used to determine blood oxygen levels associated with the brain and an internal organ.

### Modified Ratio of Ratios—Brain

Referring to FIG. 7(b), a plot of the modified ratio of ratios 703 determined from first and second signals 701, 702 derived from measured light reflected by the brain 521 of the subject 520 is shown. As illustrated, when the modified ratio of ratios plot 703 is at a minimum, the blood oxygen level is at a maximum. The modified ratio of ratios 704 when the signal value is at a maximum is indicative of the blood oxygen level being at a minimum. The minimum signal value point 708 may also occur during the A-wave component (at the end of the diastolic stage of the cardiac cycle) and the processor 562 may determine the blood oxygen level value at the minimum of the A-wave component as this may represent the tissue oxygen level. The observed decrease in the determined modified ratio of ratios during the X and Y-waves is indicative of an increase in oxygen levels during these phases of the cardiac cycle.

The processor 562 may determine that the first and second signals 701, 702 are associated with the brain 521 based on a comparison of the determined modified ratio of ratios with a characteristic waveform or a template of expected modified ratio of ratios for the brain (rather than those expected for the skin).

In some embodiments, the processor may calculate a statistical measure of the modified ratio of ratios (e.g. median, mean, or peak value) and compare it to a threshold. The statistical measure may be based on a portion of the calculated modified ratio of ratios over the waveform. For example, the statistical measure may be an average of the largest 30% of calculated modified ratio of ratios values. The statistical measure may be related to a variation or range in the determined modified ratio of ratios. As a variation in the determined modified ratio of ratios is expected from signals associated with the brain 521, the variation may be determined and compared to a threshold value. The value of the variation (or range) in determined ratio of ratio values may, for example, be greater than 1 for the processor 562 to determine that the signals are associated with the brain 521. The statistical measure may be calculated from the leading edge of the A-wave during the diastolic stage of the cardiac cycle (away from the peak signal).

This is in contrast to typical modified ratio of ratio values determined from arterial skin signals where there is little variation over the course of a pulse and has a value from about 0.5 to about 0.7. The threshold value may therefore be about 0.7 or about 1.

In some embodiments, the processor 562 may be configured to assess the health of the brain by analysing the modified ratio of ratio values. For example, the processor 562 may compare the modified ratio of ratio values to a characteristic waveform or template to make a health assessment. In some embodiments, the processor 562 may compare the statistical measure of the ratio of ratio values to the threshold to make a health assessment.

The minimum or equilibrium blood oxygen level of the brain may provide valuable clinical information about tissue oxygen levels. For example, low tissue oxygen levels in the brain are likely to be indicative of the subject 520 suffering an adverse health condition such as raised intra-cranial pressure and at risk of developing a brain injury. The processor 562 may therefore compare a statistical measure of the ratio of ratio values (or the blood oxygen level) to a threshold value, and responsive to determining that the statistical measure is less than the threshold value, the processor 562 may determine that the subject 520 is suffering from hypoxia. Brain hypoxia can occur due to stroke, vasospasm and through raised ICP.

For example, FIG. 19 shows the determined modified ratio of ratios plots from signals derived from measured light reflected by the brain of a sheep using apparatus 100. As illustrated, the determined modified ratio of ratios from signals derived from light reflected by the brain using apparatus 100 was found to increase immediately following injection of blood into the brain of sheep to thereby increase the intra-cranial pressure. This may be indicative of a drop in blood oxygen levels of the brain (and fall in cerebral perfusion) following the increase in intra-cranial pressure. A low blood oxygen level in the brain may therefore be indicative of a disorder resulting in reduced blood flow. Following the initial increase in the modified ratio of ratios a drop associated with increased blood oxygen levels was observed. This may be due to a subsequent increase in blood pressure to restore blood flow.

Accordingly, the processor 562 may compare the determined minimum blood oxygen level with a threshold level. Responsive to determining that the minimum blood oxygen level is less than the threshold level, the processor may determine that the subject 520 is suffering from organ ischaemia and that urgent treatment is required.

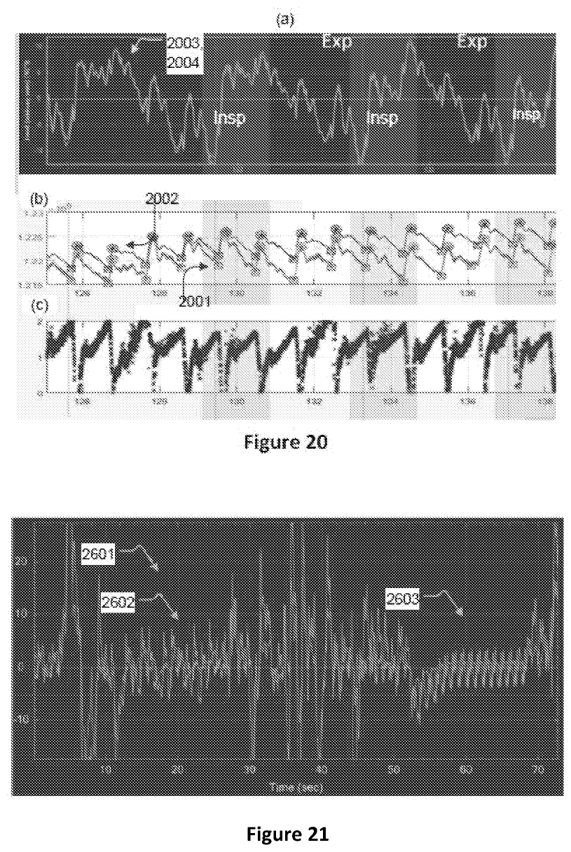

FIG. 20(b) shows signals 2001, 2002 derived from light reflected by the brain 521 of a human subject 520 using apparatus 100. As illustrated, the light intensity (or signal value) of the first and second signals 2001, 2002 derived from the brain 521 of the human subject 520 varies depending on the respiratory cycle of the subject 520. FIG. 20(a) shows simultaneous third and fourth signals 2003, 2004 derived from light reflected by the internal jugular vein of the subject 520, which provides a method to indicate the phase of the respiratory cycle. ‘Exp’ indicates the respiratory phase of expiration. ‘Insp’ indicates the respiratory phase of inspiration. As illustrated, the intensity of the third and fourth signals 2003, 2004 also varies depending on the respiratory cycle of the subject 520 and generally decreases during the expiration phase (e.g. breathing out) of the respiratory cycle and increases during inspiration phase (e.g. breathing in) of the cycle. The simultaneous changes in the modified ratio of ratios is demonstrated during each cardiac cycle is demonstrated in FIG. 20(c). Oxygen levels are shown to fall during expiration (increase in modified ratio of ratios).

As discussed above, the modified ratio of ratios (or blood oxygen levels) vary with the phase of the respiratory cycle in an internal organ. Oxygen levels may be higher during the inspiratory period. These changes reflect the extent of oxygenation of the arterial blood by the lungs during the inspiratory and expiratory phases and therefore enable lung function to be monitored. Applications of this include detection of lung injury, adjustment of mechanical ventilation to establish the best positive end expiratory pressure (PEEP) level, and adjustment of mechanical ventilation to reduce ventilator associated lung injury by selection of the lowest ventilation pressure levels that still provides adequate organ tissue oxygen levels.

### Modified Ratio of Ratios—Lung

The lungs have a unique microcirculation in that the surrounding tissues are composed of alveolar sacs that fill with oxygen during inspiration. The pulmonary arteries deliver mixed venous blood from the body to the lungs' microcirculation where oxygen is added to the blood from the alveolar sacs. Oxygen levels in the lung microvascular blood are therefore low during the periods of high blood flow into the lung that occur during systole and the diastolic recoil of the pulmonary artery (following the X-wave and Y wave components, peaks in the signal 1001. Peak oxygen levels are expected to occur during the A-wave component (during late diastole). In the lungs the minimum (trough) oxygen levels during systole provide an estimate of the mixed venous blood oxygen levels. The peak oxygen levels during diastole provide a measure of the extent of oxygenation of the blood in the lung.

FIG. 10(b) shows the calculated modified ratio of ratios from the first and second signals 1001, 1002 associated with a well ventilated lung. The modified ratio of ratios is observed to rise from 0 to up to a value of over 2 during the cardiac cycle. As illustrated, the minimum in the calculated modified ratio of ratios (which is indicative of the maximum blood oxygen value) is observed to occur during the A-wave component of the first signal 1001 (during diastole) coinciding with the timing of an additional peak 1005. This maximum blood oxygen value may provide an indication of the lung function, in terms of the extent of oxygenation of the blood. The maximum in the modified ratio of ratios (indicative of the minimum blood oxygen value) occurs during the X-wave component of the signal 1001 during the systolic stage of the cardiac cycle. The minimum blood oxygen value may provide an estimate of mixed venous blood oxygen values of blood entering the lung.

FIG. 15(c) shows the calculated modified ratio of ratios from the first and second signals 1501, 1502 associated with a dependent and both poorly ventilated and perfused lung. As illustrated, the modified ratio of ratios remains high (at around a value of about 1) and therefore blood oxygen levels remain low during the A-wave of diastole in contrast to the observed behaviour in a well ventilated lung (see FIG. 10(c)). In effect, blood is shunting through the lung without oxygen being added to the blood.

The processor 562 may determine that the first and second signals 1001, 1002, 1501, 1502 are associated with the lung based on the determined modified ratio of ratios. This may be due to the positioning of the apparatus 100 on the subject. The apparatus 100 may be adjusted until the desired signals associated with the lung are obtained. The characteristic feature of the lung is that oxygen levels are low during systole and increase during diastole (See FIG. 10(b)). This is the reverse of the skin oxygen levels. This feature may be used to determine that the signal is arising from the lungs.

In some embodiments, the processor 562 may determine that the first and second signals 1001, 1002, 1501, 1502 are associated with the lung based on the modified ratio of ratios R determined during the leading edge of the A-wave (indicative of the peak oxygen level in the lung blood) prior to the X-wave, or during the X-wave (during systole when blood oxygen levels are low).

The processor 562 may assess the health of the lung by analysing the modified ratio of ratios. For example, the processor 562 may compare the modified ratio of ratios to a characteristic waveform or template to make a health assessment. In lung injury the extent of oxygen added to the venous blood may be low. Therefore, if the modified ratio of ratios is high, this may be indicative of a lung injury.

In some embodiments, responsive to determining that the modified ratio of ratios decreases slowly to a moderate level (indicative of a slow increase of blood oxygen levels to a low or moderate level), the processor 562 determines that the subject has an unhealthy and/or poorly ventilated lung.

In some embodiments, the processor 562 may compare the statistical measure of the modified ratio of ratios to a threshold to make a health assessment. For example, responsive to determining that the modified ratio of ratios does not reach arterial oxygen levels during diastole (modified ratio of ratios of 0.5), the processor 562 determines that the subject has an unhealthy and/or poorly ventilated lung.

In some embodiments, responsive to determining that the average or median modified ratio of ratios is between about 1 and about 1.5, the processor may determine that the subject has an unhealthy or poorly ventilated lung.

### Modified Ratio of Ratios—Liver

First and second signals derived from light reflected by a healthy liver of a subject 520 using apparatus 100 may be obtained and the modified ratio of ratios from the first and second signals associated with the liver can be calculated. The modified ratio of ratio decreases during the X-wave (indicative of an increase in oxygen levels) and increases thereafter to remain relatively high and constant level through the diastolic stage of the cardiac cycle until the next X-wave. A change in this behaviour, for instance a fall in oxygen levels in the blood, may indicate a disorder of the liver with reduced blood flow such as of any one or more of: ischaemic hepatitis, abdominal compartment syndrome, and portal vein thrombosis.

The processor 562 may determine that the first and second signals are associated with the liver based on the determined modified ratio of ratios. A statistical measure of the modified ratio of ratios (e.g. median or mean) may be compared to a threshold. The statistical measure may be based on the largest 30% of calculated modified ratio of ratios over the waveform. For example, responsive to determining that the statistical measure is greater than about 0.7, the processor 562 may determine that the first and second signals 2201, 2202 are associated with the liver. In some embodiments, responsive to determining that the statistical measure is about 1, the processor 562 may determine that the first and second signals 2201, 2202 are associated with the liver. In some embodiments, responsive to determining that the statistical measure is greater than about 1, the processor 562 may determine that the first and second signals are associated with the liver.

A statistical measure equal to or less than 0.7 may be indicative of the first and second signals being associated with the skin of the subject. In some embodiments, responsive to determining that a statistical measure is equal to about 0.7, the processor 562 determines that the first and second signals are associated with the skin of the subject 520.

In some embodiments, the processor 562 may determine that the first and second signals are associated with the liver based on the modified ratio of ratios determined during the leading edge of the A-wave prior to the X-wave. This is of particular interest as the blood oxygen level (which may be calculated from the modified ratio of ratios) at this time may be similar to or equal to the tissue oxygen level of the liver.

The processor 562 may assess the health of the liver by analysing the modified ratio of ratios. For example, the processor 562 may compare the modified ratio of ratios to a characteristic waveform or template to make a health assessment. In some embodiments, the processor 562 may compare the statistical measure of the ratio of ratios to a threshold to make a health assessment. For example, responsive to determining that the statistical measure is between about 0.5 and 1.5, the processor 562 may determine that the subject has a healthy liver. In some embodiments, responsive to determining that the statistical measure is greater than about 2, the processor 562 may determine that the subject has an unhealthy liver (e.g. due to hypoxia or an ischemic liver).

### Modified Ratio of Ratios—Intestine

First and second signals derived from light reflected by healthy intestines of a subject 520 using apparatus 100 can be utilised to calculate the modified ratio of ratios from the first and second signals associated with the intestine. Oxygen levels are similar in the systolic and diastolic phases with only a brief increase (illustrated by the decrease in the calculated modified ratio of ratios) during the X-wave and systole. There is a rapid fall in oxygen levels thereafter and levels plateau during diastole—The relatively stable and low oxygen levels throughout the pulse may reflect the unique blood flow through the microcirculation of the intestinal villi. A counter current design provides liberal oxygen exchange between the arterial and venous sides of the microcirculation during all phases of the cardiac cycle.

The processor 562 may determine that the first and second signals are associated with the intestine based on the determined modified ratio of ratios. A statistical measure of the modified ratio of ratios (e.g. median or mean) may be compared to a threshold. The statistical measure may be based on the largest 30% of calculated modified ratio of ratios over the waveform. For example, responsive to determining that the statistical measure is greater than about 0.7, the processor 562 may determine that the first and second signals are associated with the intestine. In some embodiments, responsive to determining that the statistical measure is greater than about 1, the processor 562 may determine that the first and second signals are associated with the intestine. A statistical measure equal to or less than 0.7 may be indicative of the first and second signals being associated with the skin of the subject.

In some embodiments, the processor 562 may determine that the first and second signals are associated with the intestine based on the modified ratio of ratios determined during the leading edge of the A-wave prior to the X-wave.

In some embodiments, the processor 562 may determine the health condition of intestines based on a comparison of one or more waveforms of the first and second signal with a characteristic waveform. Differences in the waveform or calculated modified ratio of ratios compared to a characteristic waveform may be used to diagnose disorders of the intestine such as intestinal ischaemia which would result in very low blood oxygen levels in the intestine.

The processor 562 may assess the health of the intestines by analysing the modified ratio of ratio values. For example, the processor 562 may compare the modified ratio of ratio values to a characteristic waveform or template to make a health assessment. In some embodiments, the processor 562 may compare the statistical measure of the ratio of ratios to a threshold to make a health assessment. For example, responsive to determining that the statistical measure is greater than 2, the processor may determine that the subject has unhealthy ischaemic intestines.

### Modified Ratio of Ratios—Kidney

The modified ratio of ratio values R from signals derived from light reflected by the kidney of a subject can be calculated. Low values for the modified ratio of ratios R for the majority of the time indicate that blood oxygen levels remain very high in the kidney.

### Modified Ratio of Ratios—Muscle

The modified ratio of ratios R from signals derived from light reflected by the calf muscle at rest can be calculated. Low values for the modified ratio of ratio values R for the majority of the time indicate that blood flow to the muscle is low while it is at rest.

The invention will now be further described with reference to the following non-limiting example:

### Example—Assessment of Modifications of Brain Sensor Apparatus to Improve Detection of a Brain Optical Pulse

### Method

Variations in brain sensor apparatus design were assessed to improve detection of a brain pulse and to provide a comparison against a prior art arrangement. Assessments were undertaken in 5 healthy volunteers. Two assessments were undertaken in each volunteer, one on the left temple and one on the right, so that a total of 10 assessments were performed for each apparatus modification. The outcome measure was detection of a pulsatile optical signal consistent with the shape and characteristics of a brain pulse and the results shown in FIGS. 23 (a), (b) and (c) and in FIG. 24 are reported as the percentage of tests in which a brain pulse was detected. In each test the photo-detector (PD) and light emitting diode (LED) were circular and 8 mm in diameter. The photo-detector and LEDs (emitting light at wavelengths of 660 nm and 895 nm) were equivalent to the photo detector and LED used in the Nellcor™ Maxfast forehead sensor produced by Medtronic (710 Medtronic Parkway, Minneapolis, Minn. 55432-5604, USA). The total optical power used (both LEDS) was approximately 200 μW.

In the first trial, variations in a conventional pulse oximetry device where tested, where the photo-detector (PD) and light emitting diode (LED) were each in contact with the skin of the subject. In this trial the separation between the centre point of the light source (LED) and photo-detector (PD) were varied between 10 mm, 15 mm, 20 mm and 40 mm.

The results in FIG. 23 (a) show that using modifications of a prior art pulse oximetry device with varied spacing between PD and LED, it was not possible to detect a brain pulse signal in the case where there was no spacing between the PD/LED and the skin of the subject.

In the second trial, the spacing from the skin of the PD and LED were kept constant at 10 mm and the effect of varying the separation between the centre point of the light source (LED) and photo-detector (PD) between 10 mm, 15 mm and 20 mm was tested.

The results in FIG. 23 (b) show that while a brain pulse was detected in 50% of tests when the PD and LED were laterally separated at 10 mm and 20 mm from their centres, a separation of 15 mm between PD and LED provided 100% brain pulse detection.

In the third trial, the separation between the centre point of the light source (LED) and photo-detector (PD) was fixed at 15 mm (the optimal separation from the second trial) and the effect of varying the spacing from the skin of the PD and LED between 0 mm, 5 mm, 10 mm, 15 mm and 20 mm was tested.

The results in FIG. 23 (c) show no brain pulse was detected at spacing of 0 mm and 20 mm and that a brain pulse was detected in 25% of instances at spacing of 15 mm and in 50% of instances at 5 mm spacing. The optimal result of 100% brain pulse detection was achieved at spacing of the PD and LED from the skin of 10 mm. In subsequent testing performed by the inventor (results not shown) optimal brain pulse detection of 100% was also observed at 8.5 mm spacing of the PD and LED from the skin.

In the fourth trial, the separation between the centre point of the light source (LED) and photo-detector (PD) was fixed at 10 mm, the spacing of the PD at 10 mm from the skin was fixed and the effect of varying the spacing from the skin of the LED between 15 mm and 20 mm (that is, recessed from the PD position by 5 mm and 10 mm respectively) was tested.

The results in FIG. 24 show a relatively poor brain signal detection result in the case where the LED was located 10 mm further from the skin surface than the PD, but there was 100% brain signal detection in the case where the LED was located 5 mm further from the skin surface than the PD.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. An apparatus for determining data indicative of blood oxygen levels of internal organs, the apparatus comprising:
a body comprising a contact surface for engaging with a subject in a vicinity of an internal organ of the subject;
the body defining a first recess and second recess, the first and second recesses extending from the contact surface into the body, the second recess being separate from the first recess;
a light source comprising a light emitting region located within the first recess and configured to emit light of at least two discreet wavelengths from the first recess of the body onto the internal organ; and
a photo-detector comprising a light sensitive region located within the second recess and configured to detect light received at the second recess, wherein detected light comprises emitted light reflected from a region of a subject in proximity to an internal organ;
wherein the apparatus is configured such that the light emitting region and the light sensitive region are set back from the contact surface by from about 1 mm to about 20 mm and nearest points of the light emitting region and the light sensitive region are separated from one another by from about 4 mm to about 20 mm, such that the detected light is indicative of blood oxygen levels in blood vessels of an outermost surface of the internal organ.

2. The apparatus according to claim 1, wherein the separation between the nearest points of the light emitting region and the light sensitive region is in the range of about 5 mm to about 15 mm.

3. The apparatus according to claim 2, wherein the separation is in the range of about 6 mm to about 12 mm.

4. (canceled)

5. The apparatus according to claim 1, wherein the light emitting region and the light sensitive region are set back from the contact surface by from about 7 mm to about 10 mm.

6. The apparatus according to claim 1, wherein the body further comprises:
an outer frame defining the contact surface and a cavity; and
an inner frame shaped to fit within the cavity, wherein the inner frame defines the first recess and the second recess.

7. The apparatus according to claim 1, wherein the light source is configured to emit and sense light comprising light with wavelengths within at least a first wavelength range from about 600 nm to about 750 nm and a second wavelength range from about 855 nm to about 945 nm.

8. The apparatus according to claim 7, wherein the light source is configured to emit and sense light further comprising light with wavelength within a third wavelength range from about 780 nm to about 820 nm.

9. The apparatus according to claim 1, wherein the photo-detector is configured to emit and sense light comprising light of discrete wavelengths of at least about 660 nm, about 805 nm, about 895 nm and/or about 940 nm.

10. A system for determining blood oxygen levels of internal organs comprising the apparatus according to claim 1 and a processor, wherein the apparatus and processor are connected to enable transmission of data indicative of blood oxygen levels of internal organs from the apparatus to the processor.

11. The system according to claim 10 wherein the processor comprises memory, a display and a user interface that are all coupled to the processor.

12. A method of obtaining data indicative of blood oxygen levels of an internal organ of a subject comprising:
positioning the apparatus of claim 1 on an outer surface of the subject adjacent the internal organ;
projecting light from the light source through the outer surface of the subject to the internal organ, wherein the light comprises light at two or more discrete wavelengths;
receiving light at a photo-detector of the apparatus, the received light reflected from the internal organ at two or more discrete wavelengths respectively; and
producing a first signal indicative of the intensity of light at the first wavelength and a second signal indicative of the intensity of light at the second wavelength.

13. A method of determining blood oxygen levels of an internal organ of a subject comprising:
positioning the apparatus of the system of claim 10 on an outer surface of the subject adjacent the internal organ;
projecting light from the light source through the outer surface of the subject to the internal organ, wherein the light comprises light at two or more discrete wavelengths;
receiving light at a photo-detector of the apparatus, the received light reflected from the internal organ at the two or more discrete wavelengths respectively; and
producing a first signal indicative of the intensity of light at the first wavelength and a second signal indicative of the intensity of light at the second wavelength, wherein data relating to the first and second signals is transmitted to the processor and the blood oxygen level of the internal organ is determined.

14. The method of claim 12, further comprising:
responsive to receiving an instruction indicative of the apparatus being inaccurately positioned relative to the internal organ, repositioning the apparatus relative to the internal organ based on the instruction.

15. A computer-implemented method of assessing the health of a subject, the method comprising:
positioning the apparatus of the system of claim 10 on an outer surface of the subject adjacent the internal organ;
projecting light from the light source through the outer surface of the subject to the internal organ, wherein the light comprises light at two or more discrete wavelengths;
receiving light at a photo-detector of the apparatus, the received light reflected from the internal organ at two or more discrete wavelengths respectively;
wherein data relating to the received light is transmitted to the processor and one or more signals derived from the received light is produced and determining that at least one waveform of the one or more signals is representative of a signal predominantly associated with the internal organ; and
comparing data derived from the at least one waveform with information characteristic of a health condition to assess the health of the subject.

16. The method of claim 13, further comprising:
responsive to receiving an instruction indicative of the apparatus being inaccurately positioned relative to the internal organ, repositioning the apparatus relative to the internal organ based on the instruction.
